(19) United States
(12) Patent Application Publication
(10) Pub. No.: US 2026/0265528 A1
(43) Pub. Date: Sep. 10, 2026

(54) PIGMENTS AND RELATED METHODS

(71) Applicant: Kebotix, Inc., Cambridge, MA (US)

(72) Inventors: John Thomas Ferguson, Woburn, MA (US); Semion Saykin, Arlington, MA (US); Dennis Sheberla, Bedford, MA (US); Selma Duhovic, Brighton, MA (US); Grit Kupgan, Northville, MI (US); Victoria Campbell, Somerville, MA (US); Zachary Xavier Giustra, North Andover, MA (US); Sreeletha Joby Eldo, Andover, MA (US); David Lee Wheeler, Tewksbury, MA (US)

(73) Assignee: Kebotix, Inc., Cambridge, MA (US)

(21) Appl. No.: 19/557,194

(22) Filed: Mar. 5, 2026

Related U.S. Application Data
(60) Provisional application No. 63/767,797, filed on Mar. 6, 2025.

(51) Int. Cl. C09B 33/153; C07C 245/20; C09B 35/033

(52) U.S. Cl. CPC C09B 33/153; C07C 245/20; C09B 35/033

(57) ABSTRACT

Pigments and related methods are generally described. In some embodiments, the pigments comprise azo pigments. According to some embodiments, the pigment is non-halogenated. In certain embodiments, the method comprises a method of forming a pigment (e.g., an azo pigment). For example, in some cases, the method comprises reacting a primary amine and a nitrite ester (e.g., tert-butyl nitrite) in the presence of an acid (e.g., p-toluenesulfonic acid) and an organic solvent (e.g., ethyl acetate) to form a diazonium salt. In certain instances, the method comprises forming a pigment by reacting the diazonium salt with a coupler (e.g., in a biphasic mixture comprising the organic solvent and water). According to some embodiments, the method does not involve any halogenated compounds. In accordance with certain embodiments, the method results in reduced toxic (e.g., carcinogenic) byproducts and/or toxic (e.g., carcinogenic) degradation products.

### RELATED APPLICATIONS

This application claims priority under 35 U.S.C. § 119 (e) of U.S. Provisional Patent Application No. 63/767,797, filed Mar. 6, 2025, entitled “Pigments and Related Methods,” which is hereby incorporated herein by reference in its entirety.

### GOVERNMENT SPONSORSHIP

This invention was made with Government support under Contract No. 68HERC23C0008 awarded by the Environmental Protection Agency. The Government has certain rights in the invention.

### TECHNICAL FIELD

Pigments and related methods are generally described.

### BACKGROUND

The formation of pigments (e.g., azo pigments) may result in problematic byproducts. For example, polychlorinated biphenyl species are a toxic byproduct with carcinogenic potential that is frequently present in azo pigment samples in trace amounts on the order of parts per billion or even parts per million. Polychlorinated biphenyl species may be generated through a variety of unintended side-reactions during pigment production. For example, some methods of forming azo pigments are prone to generate polychlorinated biphenyl species, because they use hydrochloric acid in diazotization and/or rely on chlorinated aromatic solvents in post-processing to condition pigment products into the desired morphology. Polychlorinated biphenyl species may also be generated from the natural degradation of pigments. For example, many yellow azo pigments have polychlorinated structures that can degrade directly into polychlorinated biphenyl species.

Accordingly, improved pigments and/or methods of forming pigments are needed.

### SUMMARY

Pigments and related methods are generally described. In some embodiments, the pigments comprise azo pigments. According to some embodiments, the pigment is non-halogenated. In certain embodiments, the method comprises a method of forming a pigment (e.g., an azo pigment). For example, in some cases, the method comprises reacting a primary amine and a nitrite ester (e.g., tert-butyl nitrite) in the presence of an acid (e.g., p-toluenesulfonic acid) and an organic solvent (e.g., ethyl acetate) to form a diazonium salt. In certain instances, the method comprises forming a pigment by reacting the diazonium salt with a coupler (e.g., in a biphasic mixture comprising the organic solvent and water). According to some embodiments, the method does not involve any halogenated compounds. In accordance with certain embodiments, the method results in reduced toxic (e.g., carcinogenic) byproducts and/or toxic (e.g., carcinogenic) degradation products. The subject matter of the present disclosure involves, in some cases, interrelated products, alternative solutions to a particular problem, and/or a plurality of different uses of one or more systems and/or articles.

Some aspects relate to methods of forming a pigment. In some embodiments, the method comprises reacting a primary amine and a nitrite ester in the presence of an acid and an organic solvent to form a diazonium salt; and forming a pigment by reacting the diazonium salt with a coupler in a biphasic mixture comprising the organic solvent and water.

In certain embodiments, the method comprises reacting a primary amine and a nitrite ester in the presence of an acid and an organic solvent to form a diazonium salt; and forming a pigment by reacting the diazonium salt with a coupler; wherein the method does not involve any halogenated compounds; and wherein the pigment comprises an azo pigment.

Some aspects relate to pigments. In some embodiments, the pigment comprises:

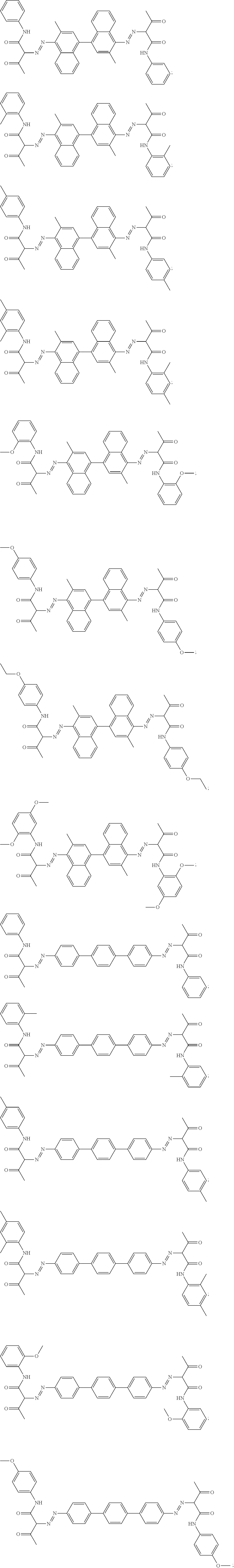

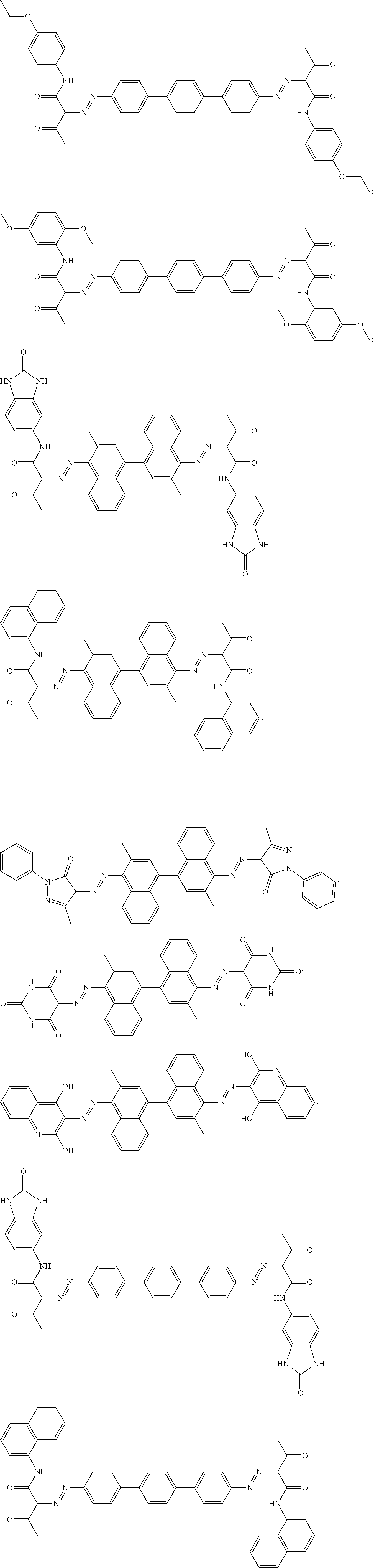

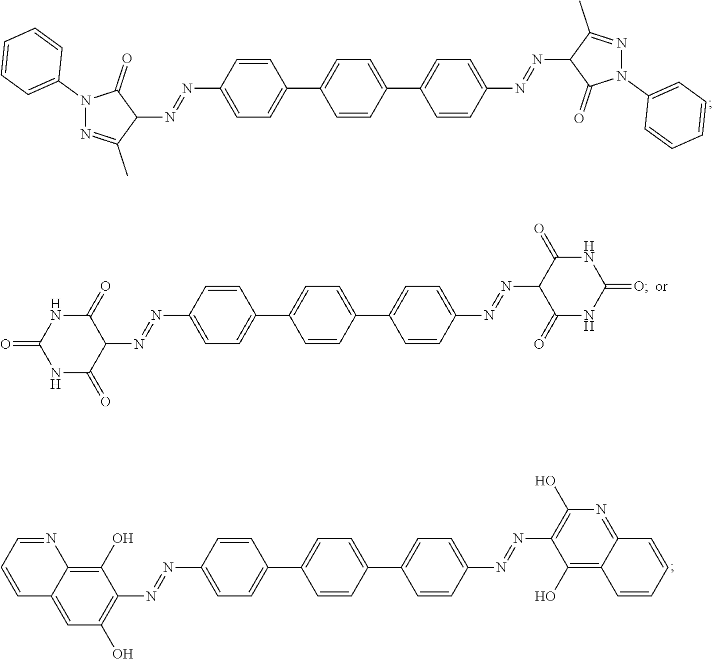

or a tautomer or polymorph thereof.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### DETAILED DESCRIPTION

Pigments and related methods are generally described. In some embodiments, the pigments comprise azo pigments. According to some embodiments, the pigment is non-halogenated. In certain embodiments, the method comprises a method of forming a pigment (e.g., an azo pigment). For example, in some cases, the method comprises reacting a primary amine and a nitrite ester (e.g., tert-butyl nitrite) in the presence of an acid (e.g., p-toluenesulfonic acid) and an organic solvent (e.g., ethyl acetate) to form a diazonium salt. In certain instances, the method comprises forming a pigment by reacting the diazonium salt with a coupler (e.g., in a biphasic mixture comprising the organic solvent and water). According to some embodiments, the method does not involve any halogenated compounds. In accordance with certain embodiments, the method results in reduced toxic (e.g., carcinogenic) byproducts and/or toxic (e.g., carcinogenic) degradation products.

Certain aspects are related to methods. In some embodiments, the method comprises a method of forming a pigment.

In certain embodiments, the method comprises reacting a primary amine and a nitrite ester. For example, in some cases, the method comprises reacting a primary amine and a nitrite ester to form a diazonium salt. As another example, in certain instances, the method comprises reacting a primary amine and a nitrite ester in the presence of an acid and/or an organic solvent to form a diazonium salt. For example, according to some embodiments, the method comprises reacting a primary amine and a nitrite ester in the presence of an acid and an organic solvent to form a diazonium salt.

In some cases, the reaction proceeds relatively quickly after the primary amine and nitrite ester are combined in the presence of an acid and/or an organic solvent. For example, in certain instances, the diazonium salt is completely or substantially completely (e.g., at least 70%, at least 80%, at least 90%, or 100% of the theoretical yield) formed in greater than or equal to 1 minute, greater than or equal to 5 minutes, greater than or equal to 10 minutes, greater than or equal to 20 minutes, greater than or equal to 30 minutes, greater than or equal to 45 minutes, greater than or equal to 1 hour, greater than or equal to 2 hours, greater than or equal to 3 hours, greater than or equal to 4 hours, or greater than or equal to 5 hours after the primary amine and nitrite ester are combined in the presence of an acid and/or an organic solvent. According to some embodiments, the diazonium salt is completely or substantially completely (e.g., at least 70%, at least 80%, at least 90%, or 100% of the theoretical yield) formed in less than or equal to 3 days, less than or equal to 2 days, less than or equal to 24 hours, less than or equal to 22 hours, less than or equal to 20 hours, less than or equal to 18 hours, less than or equal to 16 hours, less than or equal to 14 hours, less than or equal to 12 hours, less than or equal to 10 hours, less than or equal to 8 hours, less than or equal to 6 hours, less than or equal to 5 hours, less than or equal to 4 hours, less than or equal to 3 hours, less than or equal to 2 hours, less than or equal to 1 hour, less than or equal to 45 minutes, or less than or equal to 30 minutes after the primary amine and nitrite ester are combined in the presence of an acid and/or an organic solvent. Combinations of these ranges are also possible (e.g., greater than or equal to 1 minute and less than or equal to 3 days, greater than or equal to 5 minutes and less than or equal to 1 hour, or greater than or equal to 5 minutes and less than or equal to 30 minutes).

In accordance with certain embodiments, the primary amine lacks an electron withdrawing group. Without wishing to be bound by theory, it is believed that electron withdrawing groups on the primary amine may hinder reactivity and/or slow down reaction times, in some cases. Moreover, without wishing to be bound by theory, it is believed that excess of the acid and/or nitrite ester may increase reactivity and/or speed up reaction times, counteracting the effects of electron withdrawing groups on the primary amine, in some instances.

According to some embodiments, the primary amine comprises one or two primary amine groups. For example, in certain cases, the primary amine comprises one primary amine group. As another example, in some instances, the primary amine comprises two primary amine groups.

In some cases, the method comprises suspending and/or dissolving the primary amine in the organic solvent prior to reacting with the nitrite ester. For example, in certain instances, the method comprises dissolving the primary amine in the organic solvent prior to reacting with the nitrite ester.

According to some embodiments, the nitrite ester comprises methyl nitrite, ethyl nitrite, n-propyl nitrite, isopropyl nitrite, n-butyl nitrite, sec-butyl nitrite, and/or tert-butyl nitrite. For example, in some instances, the nitrite ester comprises tert-butyl nitrite.

The nitrite ester and primary amine may be present in a suitable ratio. For example, in certain embodiments, the molar ratio of the nitrite ester to the primary amine is greater than or equal to 1:1 (e.g., greater than 1:1), greater than or equal to 1.1:1, greater than or equal to 1.2:1, greater than or equal to 1.3:1, greater than or equal to 1.4:1, greater than or equal to 1.5:1, greater than or equal to 1.6:1, greater than or equal to 1.7:1, greater than or equal to 1.8:1, greater than or equal to 1.9:1, greater than or equal to 2:1 (e.g., greater than 2:1), greater than or equal to 2.1:1, greater than or equal to 2.2:1, greater than or equal to 2.3:1, greater than or equal to 2.4:1, or greater than or equal to 2.5:1. In some embodiments, the molar ratio of the nitrite ester to the primary amine is less than or equal to 5:1, less than or equal to 4.5:1, less than or equal to 4:1, less than or equal to 3.5:1, less than or equal to 3:1, less than or equal to 2.9:1, less than or equal to 2.8:1, less than or equal to 2.7:1, less than or equal to 2.6:1, less than or equal to 2.5:1, less than or equal to 2.4:1, less than or equal to 2.3:1, less than or equal to 2.2:1, less than or equal to 2.1:1, less than or equal to 2:1, less than or equal to 1.9:1, less than or equal to 1.8:1, less than or equal to 1.7:1, less than or equal to 1.6:1, less than or equal to 1.5:1, less than or equal to 1.4:1, less than or equal to 1.3:1, less than or equal to 1.2:1, or less than or equal to 1.1:1. Combinations of these ranges are also possible (e.g., greater than or equal to 1:1 and less than or equal to 5:1, greater than 1:1 and less than or equal to 5:1, greater than 1:1 and less than or equal to 3:1, greater than 1:1 and less than or equal to 2.1:1, greater than 1:1 and less than or equal to 1.1:1, greater than or equal to 2:1 and less than or equal to 5:1, greater than 2:1 and less than or equal to 5:1, greater than 2:1 and less than or equal to 3:1, or greater than 2:1 and less than or equal to 2.1:1).

In some cases where the primary amine comprises one primary amine group, the molar ratio of the nitrite ester to the primary amine is greater than or equal to 1:1 (e.g., greater than or equal to 1:1 and less than or equal to 5:1, greater than 1:1 and less than or equal to 5:1, greater than 1:1 and less than or equal to 3:1, greater than 1:1 and less than or equal to 2.1:1, or greater than 1:1 and less than or equal to 1.1:1). In certain cases where the primary amine comprises two primary amine groups, the molar ratio of the nitrite ester to the primary amine is greater than or equal to 2:1 (e.g., greater than or equal to 2:1 and less than or equal to 5:1, greater than 2:1 and less than or equal to 5:1, greater than 2:1 and less than or equal to 3:1, or greater than 2:1 and less than or equal to 2.1:1).

In certain embodiments, the acid comprises an organic acid. In some cases, the acid comprises a strong acid. In certain instances, the acid comprises a monoprotic acid. According to some embodiments, the acid comprises an organic acid and/or a strong acid and/or a monoprotic acid. For example, in some instances, the acid comprises a monoprotic strong organic acid. In accordance with some embodiments, the acid is non-halogenated. In certain cases, the acid and/or method does not include HCl.

In accordance with certain embodiments, the acid (e.g., the monoprotic strong organic acid) comprises an aromatic sulfonic acid. Non-limiting examples of aromatic sulfonic acids include benzenesulfonic acid and/or p-toluenesulfonic acid. For example, in some cases, the acid (e.g., the monoprotic strong organic acid) comprises p-toluenesulfonic acid.

The acid and primary amine may be present in a suitable ratio. For example, in certain embodiments, the molar ratio of the acid to the primary amine is greater than or equal to 1:1 (e.g., greater than 1:1), greater than or equal to 1.1:1, greater than or equal to 1.2:1, greater than or equal to 1.3:1, greater than or equal to 1.4:1, greater than or equal to 1.5:1, greater than or equal to 1.6:1, greater than or equal to 1.7:1, greater than or equal to 1.8:1, greater than or equal to 1.9:1, greater than or equal to 2:1 (e.g., greater than 2:1), greater than or equal to 2.1:1, greater than or equal to 2.2:1, greater than or equal to 2.3:1, greater than or equal to 2.4:1, or greater than or equal to 2.5:1. In some embodiments, the molar ratio of the acid to the primary amine is less than or equal to 5:1, less than or equal to 4.5:1, less than or equal to 4:1, less than or equal to 3.5:1, less than or equal to 3:1, less than or equal to 2.9:1, less than or equal to 2.8:1, less than or equal to 2.7:1, less than or equal to 2.6:1, less than or equal to 2.5:1, less than or equal to 2.4:1, less than or equal to 2.3:1, less than or equal to 2.2:1, less than or equal to 2.1:1, less than or equal to 2:1, less than or equal to 1.9:1, less than or equal to 1.8:1, less than or equal to 1.7:1, less than or equal to 1.6:1, less than or equal to 1.5:1, less than or equal to 1.4:1, less than or equal to 1.3:1, less than or equal to 1.2:1, or less than or equal to 1.1:1. Combinations of these ranges are also possible (e.g., greater than or equal to 1:1 and less than or equal to 5:1, greater than 1:1 and less than or equal to 5:1, greater than 1:1 and less than or equal to 3:1, greater than 1:1 and less than or equal to 2.1:1, greater than 1:1 and less than or equal to 1.1:1, greater than or equal to 2:1 and less than or equal to 5:1, greater than 2:1 and less than or equal to 5:1, greater than 2:1 and less than or equal to 3:1, or greater than 2:1 and less than or equal to 2.1:1).

In some cases where the primary amine comprises one primary amine group, the molar ratio of the acid to the primary amine is greater than or equal to 1:1 (e.g., greater than or equal to 1:1 and less than or equal to 5:1, greater than 1:1 and less than or equal to 5:1, greater than 1:1 and less than or equal to 3:1, greater than 1:1 and less than or equal to 2.1:1, or greater than 1:1 and less than or equal to 1.1:1). In certain cases where the primary amine comprises two primary amine groups, the molar ratio of the acid to the primary amine is greater than or equal to 2:1 (e.g., greater than or equal to 2:1 and less than or equal to 5:1, greater than 2:1 and less than or equal to 5:1, greater than 2:1 and less than or equal to 3:1, or greater than 2:1 and less than or equal to 2.1:1).

In some embodiments, the acid is a solid. In certain embodiments, the acid is a liquid. In some cases, the acid is a solution (e.g., a concentrated solution). For example, in certain instances, the acid is a solution (e.g., a concentrated solution) in the organic solvent.

In accordance with certain embodiments, the method comprises adding the acid to the reaction mixture comprising the primary amine, nitrite ester, and organic solvent. In some cases, adding the acid to the reaction mixture results in one or more diazonium salts precipitating from the reaction mixture.

According to some embodiments, the organic solvent is immiscible with water. For example, in some cases, less than or equal to 25% (w/w), less than or equal to 23% (w/w), less than or equal to 20% (w/w), less than or equal to 18% (w/w), less than or equal to 15% (w/w), less than or equal to 13% (w/w), less than or equal to 10% (w/w), less than or equal to 8% (w/w), less than or equal to 5% (w/w), less than or equal to 3% (w/w), or less than or equal to 1% (w/w) of the organic solvent is soluble in water. In certain instances, greater than or equal to 0% (w/w), greater than or equal to 1% (w/w), greater than or equal to 3% (w/w), greater than or equal to 5% (w/w), greater than or equal to 7% (w/w), greater than or equal to 10% (w/w), greater than or equal to 12% (w/w), greater than or equal to 15% (w/w), or greater than or equal to 17% (w/w) of the organic solvent is soluble in water. Combinations of these ranges are also possible (e.g., greater than or equal to 0% (w/w) and less than or equal to 25% (w/w), greater than or equal to 0% (w/w) and less than or equal to 10% (w/w), or greater than or equal to 5% (w/w) and less than or equal to 10% (w/w)). In some instances, the organic solvent is 0% (w/w) soluble in water. Without wishing to be bound by theory, it is believed that using an organic solvent that is immiscible with water allows a bilayer system, which provides greater control of the reaction rate, which results in better quality pigment, in some instances. Without wishing to be bound by theory, it is also believed that using an organic solvent that is immiscible with water improves efficiency of the workup by facilitating extraction of aqueous impurities while retaining products in the organic layer, in certain cases.

In certain embodiments, the organic solvent comprises an ester. Non-limiting examples of esters include ethyl acetate, n-butyl acetate, t-butyl acetate, n-hexyl acetate, and/or n-octyl acetate. For example, in some cases, the organic solvent comprises ethyl acetate.

In some embodiments, the method comprises protecting the diazonium salt from UV light and/or bases. Without wishing to be bound by theory, it is believed that UV light and/or base may cause degradation of the diazonium salt, in certain cases.

In accordance with certain embodiments, the method comprises reacting the diazonium salt with a coupler. For example, in some cases, the method comprises forming a pigment by reacting the diazonium salt with a coupler. In some instances, forming the pigment by reacting the diazonium salt with the coupler comprises coupling the coupler to the diazonium moiety of the diazonium salt. In some cases, the method comprises forming a pigment by reacting the diazonium salt with a coupler in a biphasic mixture. In accordance with some embodiments, reacting the diazonium salt with a coupler comprises mixing the diazonium salt with the coupler.

In some cases, the reaction between the diazonium salt and coupler proceeds relatively quickly. For example, in certain instances, the pigment is completely or substantially completely (e.g., at least 70%, at least 80%, at least 90%, or 100% of the theoretical yield) formed greater than or equal to 10 minutes, greater than or equal to 30 minutes, greater than or equal to 45 minutes, greater than or equal to 1 hour, greater than or equal to 2 hours, greater than or equal to 3 hours, greater than or equal to 4 hours, greater than or equal to 5 hours, greater than or equal to 9 hours, greater than or equal to 12 hours, greater than or equal to 15 hours, greater than or equal to 18 hours, greater than or equal to 21 hours, or greater than or equal to 24 hours after the diazonium salt and coupler are combined. According to some embodiments, the pigment is completely or substantially completely (e.g., at least 70%, at least 80%, at least 90%, or 100% of the theoretical yield) formed in less than or equal to 3 days, less than or equal to 2 days, less than or equal to 36 hours, less than or equal to 24 hours, less than or equal to 22 hours, less than or equal to 20 hours, less than or equal to 18 hours, less than or equal to 16 hours, less than or equal to 14 hours, less than or equal to 12 hours, less than or equal to 10 hours, less than or equal to 8 hours, less than or equal to 6 hours, less than or equal to 5 hours, less than or equal to 4 hours, less than or equal to 3 hours, less than or equal to 2 hours, or less than or equal to 1 hour after the diazonium salt and coupler are combined. Combinations of these ranges are also possible (e.g., greater than or equal to 10 minutes and less than or equal to 3 days, greater than or equal to 1 hour and less than or equal to 3 hours, or greater than or equal to 5 hours and less than or equal to 24 hours). Without wishing to be bound by theory, it is believed that the reaction time for the formation of the pigment may be decreased by increasing the alkalinity, increasing mixing, using shear mixing surfaces, and/or adding small quantities of crystalline pigment to promote nucleation, in accordance with certain embodiments. For example, in some cases, mixing reduces the formation of agglomerates, which can hinder pigment isolation.

In certain cases, the method comprises reacting the diazonium salt with the coupler without first isolating the diazonium salt. Without wishing to be bound by theory, it is believed that reacting the diazonium salt with the coupler without first isolating the diazonium salt may reduce hazards and/or increase product yields, in some instances. For example, in some cases, isolating the diazonium salt (e.g., prior to coupling) may be hazardous due, for example, to the explosive nature of diazonium chloride intermediates. Without wishing to be bound by theory, it is believed that isolating the diazonium salt (e.g., prior to coupling) is not needed, in some embodiments of the method disclosed herein (e.g., in some embodiments wherein the method comprises reacting the diazonium salt with a coupler in a biphasic mixture comprising the organic solvent and water), as the generation of nitrous acid during diazotization is reversible and such species are rapidly removed from solution as coupling raises the pH value, such that they do not react with products unfavorably to degrade them and/or generate hazardous nitrosamines. Moreover, in certain embodiments wherein the method comprises reacting the diazonium salt with a coupler in a biphasic mixture comprising the organic solvent and water, the deleterious effects of nitrous acid on pigment products may be mitigated further by its affinity to the aqueous phase as the pigment collects separately in the organic layer.

According to certain embodiments, reacting the diazonium salt with the coupler comprises adding the diazonium salt to the coupler. In accordance with some embodiments, reacting the diazonium salt with the coupler comprises adding the coupler to the diazonium salt. In certain cases, reacting the diazonium salt with the coupler comprises adding the diazonium salt to the coupler and/or adding the coupler to the diazonium salt slowly. In some instances, reacting the diazonium salt with the coupler comprises adding the diazonium salt to the coupler and/or adding the coupler to the diazonium salt rapidly. Without wishing to be bound by theory, it is believed that, unlike in comparator methods, the methods disclosed herein, in some embodiments, are not sensitive to the order of addition (e.g., adding coupler to diazonium salt or vice versa) and/or rate of addition (e.g., slow addition versus rapid addition). For example, without wishing to be bound by theory, it is believed that, in certain embodiments of the methods disclosed herein, reactions occur in either phase of the reaction mixture as coupling species diffuse upward into the organic layer or diazonium salts settle downward into the aqueous layer, and, optionally, stirring the aqueous layer assists aqueous diffusion and maintains an excess of the coupling species at the interface.

In accordance with some embodiments, reacting the diazonium salt with the coupler comprises adding the diazonium salt to the coupler and/or adding the coupler to the diazonium salt in greater than 0 minutes, greater than or equal to 1 minute, greater than or equal to 3 minutes, greater than or equal to 5 minutes, greater than or equal to 10 minutes, greater than or equal to 15 minutes, greater than or equal to 20 minutes, greater than or equal to 30 minutes, greater than or equal to 45 minutes, greater than or equal to 1 hour, or greater than or equal to 2 hours. In certain embodiments, reacting the diazonium salt with the coupler comprises adding the diazonium salt to the coupler and/or adding the coupler to the diazonium salt in less than or equal to 24 hours, less than or equal to 22 hours, less than or equal to 20 hours, less than or equal to 18 hours, less than or equal to 15 hours, less than or equal to 12 hours, less than or equal to 9 hours, less than or equal to 6 hours, less than or equal to 3 hours, less than or equal to 2 hours, less than or equal to 1 hour, less than or equal to 45 minutes, less than or equal to 30 minutes, less than or equal to 15 minutes, or less than or equal to 5 minutes. Combinations of these ranges are also possible (e.g., greater than 0 minutes and less than or equal to 24 hours, greater than or equal to 1 minute and less than or equal to 1 hour, or greater than or equal to 1 minute and less than or equal to 15 minutes).

In accordance with certain embodiments, reacting the diazonium salt with the coupler comprises stirring. For example, in some cases reacting the diazonium salt with the coupler comprises stirring an aqueous layer of the biphasic mixture.

In some embodiments, the biphasic mixture comprises an organic solvent (e.g., any organic solvent disclosed herein) and water (e.g., pure water or an aqueous solution comprising water). The organic solvent and water may be present at a suitable ratio. For example, in certain embodiments, the organic solvent to water volumetric ratio is greater than or equal to 1:5, greater than or equal to 1:4, greater than or equal to 1:3, greater than or equal to 1:2, greater than or equal to 1:1, greater than or equal to 2:1, greater than or equal to 3:1, or greater than or equal to 4:1. In some cases, the organic solvent to water volumetric ratio is less than or equal to 5:1, less than or equal to 4:1, less than or equal to 3:1, less than or equal to 2:1, less than or equal to 1:1, or less than or equal to 1:2. Combinations of these ranges are also possible (e.g., greater than or equal to 1:5 and less than or equal to 5:1 or greater than or equal to 1:2 and less than or equal to 2:1). In certain instances, the organic solvent to water volumetric ratio is about 1:1. According to some embodiments, additional organic solvent (e.g., ratios greater than 1:1) is used when the primary amine has poor solubility and/or high molecular weight. In accordance with some embodiments, additional water (e.g., ratios less than 1:1) is used when the coupler has poor solubility and/or high molecular weight.

As used herein, a “coupler” is a compound that comprises one or more acidic carbons that can serve as a nucleophile, wherein an acidic carbon is a carbon bonded to one or more labile hydrogens, wherein the C—H bond has a pKa value of less than or equal to 30. For example, in some embodiments, the coupler comprises a C—H bond with a pKa value of less than or equal to 30, less than or equal to 28, less than or equal to 25, less than or equal to 23, less than or equal to 20, less than or equal to 18, less than or equal to 15, less than or equal to 13, less than or equal to 10, less than or equal to 8, or less than or equal to 5. In certain embodiments, the coupler comprises a C—H bond with a pKa value of greater than or equal to 1, greater than or equal to 3, greater than or equal to 5, greater than or equal to 7, greater than or equal to 10, greater than or equal to 12, greater than or equal to 15, greater than or equal to 17, greater than or equal to 20, or greater than or equal to 22. Combinations of these ranges are also possible (e.g., greater than or equal to 1 and less than or equal to 30, greater than or equal to 1 and less than or equal to 23, greater than or equal to 1 and less than or equal to 13, greater than or equal to 5 and less than or equal to 30, greater than or equal to 5 and less than or equal to 23, or greater than or equal to 5 and less than or equal to 13). Without wishing to be bound by theory, it is believed that couplers with lower pKa values have higher reactivity, all other factors being equal, in methods disclosed herein, in some cases.

The coupler may have a suitable number of acidic carbons. For example, in some cases, the coupler comprises greater than or equal to one acidic carbon, greater than or equal to two acidic carbons, greater than or equal to three acidic carbons, greater than or equal to four acidic carbons, or greater than or equal to five acidic carbons. In certain instances, the coupler comprises less than or equal to ten acidic carbons, less than or equal to eight acidic carbons, less than or equal to six acidic carbons, less than or equal to five acidic carbons, less than or equal to four acidic carbons, less than or equal to three acidic carbons, or less than or equal to two acidic carbons. Combinations of these ranges are also possible (e.g., greater than or equal to one acidic carbon and less than or equal to ten acidic carbons, greater than or equal to one acidic carbon and less than or equal to five acidic carbons, or greater than or equal to one acidic carbon and less than or equal to two acidic carbons). For example, in some cases, the coupler comprises one acidic carbon. As another example, in certain cases, the coupler comprises two acidic carbons. Each acidic carbon may independently have one, two, or three labile hydrogens.

The coupler may have a suitable number of labile hydrogens bonded to carbon. For example, in some cases, the coupler comprises greater than or equal to one labile hydrogen, greater than or equal to two labile hydrogens, greater than or equal to three labile hydrogens, greater than or equal to four labile hydrogens, greater than or equal to five labile hydrogens, greater than or equal to seven labile hydrogens, greater than or equal to ten labile hydrogens, greater than or equal to twelve labile hydrogens, or greater than or equal to fifteen labile hydrogens bonded to carbon. In certain instances, the coupler comprises less than or equal to twenty labile hydrogens, less than or equal to eighteen labile hydrogens, less than or equal to fifteen labile hydrogens, less than or equal to thirteen labile hydrogens, less than or equal to ten labile hydrogens, less than or equal to eight labile hydrogens, less than or equal to six labile hydrogens, less than or equal to five labile hydrogens, less than or equal to four labile hydrogens, less than or equal to three labile hydrogens, or less than or equal to two labile hydrogens bonded to carbon. Combinations of these ranges are also possible (e.g., greater than or equal to one and less than or equal to twenty labile hydrogens, greater than or equal to one and less than or equal to ten labile hydrogens, greater than or equal to one and less than or equal to five labile hydrogens, or greater than or equal to one and less than or equal to two labile hydrogens). For example, in some cases, the coupler comprises only one labile hydrogen bonded to carbon. As another example, in certain cases, the coupler comprises greater than or equal to two (e.g. only two) labile hydrogens bonded to carbon.

For the sake of illustration, a non-limiting example of a coupler with only one labile hydrogen bonded to carbon, in accordance with some embodiments, includes the following, wherein alpha indicates the location of the labile hydrogen bonded to carbon:

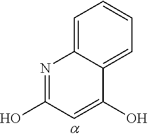 [SMILES: Oc1cc(O)c2ccccc2n1]

In instances where the coupler has greater than one labile hydrogen bonded to carbon, the labile hydrogens bonded to carbon may be equivalent or inequivalent. Labile hydrogens bonded to carbon are equivalent if they are bonded to the same acidic carbon and/or if they are identical to each other through the molecule's symmetry operations. For the sake of illustration, a non-limiting example of a coupler with two equivalent labile hydrogens bonded to carbon, in accordance with some embodiments, includes the following, wherein alpha indicates the location of the equivalent labile hydrogens bonded to carbon:

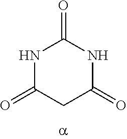

For the sake of illustration, non-limiting examples of couplers with two or more inequivalent labile hydrogens bonded to carbon, in accordance with some embodiments, include the following, wherein the alphas indicate the locations of the labile hydrogens bonded to carbon and the numbering of the alphas indicates whether they are equivalent or inequivalent:

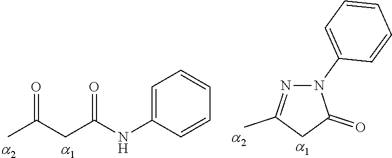

For the sake of illustration, a non-limiting example of a coupler with two or more equivalent labile hydrogens (in different sites) bonded to carbon and two or more inequivalent labile hydrogens (in different sites) bonded to carbon, in accordance with some embodiments, is shown as follows, wherein the alphas indicate the locations of the labile hydrogens bonded to carbon and the numbering of the alphas indicates whether they are equivalent or inequivalent:

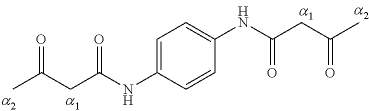 [SMILES: CC(=O)CC(=O)Nc1ccc(NC(=O)CC(C)=O)cc1]

In some cases, the method comprises using different conditions for couplers having different numbers of labile hydrogens bonded to carbon and/or different types (e.g., equivalent or inequivalent) of labile hydrogens bonded to carbon. For example, in certain instances, the reaction conditions (e.g., dissolution in alkaline solutions) for couplers having only one labile hydrogen bonded to carbon or couplers having two or more labile hydrogens bonded to carbon wherein each labile hydrogen bonded to carbon is equivalent (e.g., bonded to the same carbon) may be selected to provide a rapid reaction time and/or ease of processability, while the reaction conditions (e.g., acidic slurries with mixing) for couplers having two or more inequivalent labile hydrogen bonded to carbon (e.g., in proximate sites) may be selected to provide a homogeneous (or substantially homogeneous) product distribution and/or decrease byproduct formation. Without wishing to be bound by theory, it is believed that increasing the pH of the coupler combination, dispersion, solution and/or slurry may shorten reaction times and/or increase the ease of processability but may facilitate byproduct formation, in some instances.

In certain embodiments where the coupler comprises only one labile hydrogen bonded to carbon and/or only equivalent labile hydrogens (e.g., two equivalent labile hydrogens) bonded to carbon (e.g., bonded to the same carbon), the method comprises combining (e.g., dispersing and/or dissolving) at least a portion (e.g., at least 10%, at least 25%, at least 50%, at least 75%, at least 90%, or all) of the coupler with an alkaline solution (e.g., a solution having a pH of greater than 7, a pH of greater than 7.5, a pH of greater than 8, a pH of greater than 9, a pH of greater than 10, a pH of greater than 11, a pH of greater than 12, or a pH of greater than 13; a pH of less than or equal to 14; a pH of less than 13, a pH of less than 12, a pH of less than 11, a pH of less than 10, a pH of less than 9, or a pH of less than 8; or combinations thereof, such as a pH of greater than 7 and less than or equal to 14). In some cases, combining at least a portion of the coupler with an alkaline solution forms a solution, slurry, dispersion, and/or emulsion.

According to some embodiments where the coupler comprises two or more inequivalent labile hydrogens bonded to carbon, the method comprises combining (e.g., dispersing and/or dissolving) at least a portion (e.g., at least 10%, at least 25%, at least 50%, at least 75%, at least 90%, or all) of the coupler with an acidic solution (e.g., a solution having a pH of less than 7, a pH of less than 6.5, a pH of less than 6, a pH of less than 5.5, a pH of less than 5, a pH of less than 4.5, a pH of less than 4, a pH of less than 3, or a pH of less than 2; a pH of greater than or equal to 1, a pH of greater than 2, a pH of greater than 3, a pH of greater than 4, a pH of greater than 4.5, a pH of greater than 5, a pH of greater than 5.5, or a pH of greater than 6; or combinations thereof, such as a pH of less than 7 and greater than or equal to 1 or a pH of less than 6 and greater than 4) (e.g., a buffered solution with moderate acidity). In some cases, combining at least a portion of the coupler with an acidic solution forms a solution, slurry, dispersion, and/or emulsion. For example, in certain instances, combining at least a portion of the coupler with an acidic solution forms a slurry and/or dispersion that is diluted (e.g., with the organic solvent) to obtain emulsions (e.g., for better flowability).

The coupler and the diazonium salt may be present in a suitable ratio. For example, in certain embodiments, the molar ratio of the coupler to the diazonium salt is greater than or equal to 1:2 (e.g., greater than 1.2), greater than or equal to 3:4, greater than or equal to 1:1 (e.g., greater than 1:1), greater than or equal to 1.1:1, greater than or equal to 1.2:1, greater than or equal to 1.3:1, greater than or equal to 1.4:1, greater than or equal to 1.5:1, greater than or equal to 1.6:1, greater than or equal to 1.7:1, greater than or equal to 1.8:1, greater than or equal to 1.9:1, greater than or equal to 2:1 (e.g., greater than 2.1), greater than or equal to 2.1:1, greater than or equal to 2.2:1, greater than or equal to 2.3:1, greater than or equal to 2.4:1, or greater than or equal to 2.5:1. In some embodiments, the molar ratio of the coupler to the diazonium salt is less than or equal to 5:1, less than or equal to 4.5:1, less than or equal to 4:1, less than or equal to 3.5:1, less than or equal to 3:1, less than or equal to 2.9:1, less than or equal to 2.8:1, less than or equal to 2.7:1, less than or equal to 2.6:1, less than or equal to 2.5:1, less than or equal to 2.4:1, less than or equal to 2.3:1, less than or equal to 2.2:1, less than or equal to 2.1:1, less than or equal to 2:1, less than or equal to 1.9:1, less than or equal to 1.8:1, less than or equal to 1.7:1, less than or equal to 1.6:1, less than or equal to 1.5:1, less than or equal to 1.4:1, less than or equal to 1.3:1, less than or equal to 1.2:1, or less than or equal to 1.1:1. Combinations of these ranges are also possible (e.g., greater than or equal to 1:2 and less than or equal to 5:1, greater than 1:2 and less than or equal to 5:1, greater than 1:1 and less than or equal to 5:1, greater than 1:1 and less than or equal to 3:1, greater than 1:1 and less than or equal to 2.1:1, greater than 1:1 and less than or equal to 1.1:1, greater than or equal to 2:1 and less than or equal to 5:1, greater than 2:1 and less than or equal to 5:1, greater than 2:1 and less than or equal to 3:1, or greater than 2:1 and less than or equal to 2.1:1).

For example, in certain cases where a coupler reacts 1:1 with the diazonium salt (e.g., where the diazonium salt comprises one diazonium group and the coupler comprises only one acidic carbon which is more acidic than all other acidic carbons), the molar ratio of the coupler to the diazonium salt is greater than or equal to 1:1 (e.g., greater than or equal to 1:1 and less than or equal to 5:1, greater than 1:1 and less than or equal to 5:1, greater than 1:1 and less than or equal to 3:1, greater than 1:1 and less than or equal to 2.1:1, or greater than 1:1 and less than or equal to 1.1:1). Without wishing to be bound by theory, it is believed that, in some instances, when a coupler reacts 1:1 with the diazonium salt, a slight excess of the coupler maximizes yield while maintaining lower reagent costs, and that less excess is needed with mixing.

As another example, in some cases where a coupler reacts 2:1 with the diazonium salt (e.g., where the diazonium salt comprises two diazonium groups and the coupler comprises only one only one acidic carbon which is more acidic than all other acidic carbons), the molar ratio of the coupler to the diazonium salt is greater than or equal to 2:1 (e.g., greater than or equal to 2:1 and less than or equal to 5:1, greater than 2:1 and less than or equal to 5:1, greater than 2:1 and less than or equal to 3:1, or greater than 2:1 and less than or equal to 2.1:1). Without wishing to be bound by theory, it is believed that, in some instances, when a coupler reacts 2:1 with the diazonium salt, a moderate excess of the coupler maximizes yield while maintaining lower reagent costs, and that less excess is needed with mixing.

As another example, in certain instances where a coupler reacts 1:2 with the diazonium salt (e.g., where the diazonium salt comprises one diazonium group and the coupler comprises two acidic carbons which are tied for most acidic), the molar ratio of the coupler to the diazonium salt is greater than or equal to 1:2 (e.g., greater than or equal to 1:2 and less than or equal to 5:1, greater than 1:2 and less than or equal to 5:1, greater than 1:1 and less than or equal to 5:1, greater than 1:1 and less than or equal to 3:1, greater than 1:1 and less than or equal to 2.1:1, or greater than 1:1 and less than or equal to 1.1:1). Without wishing to be bound by theory, it is believed that, in certain cases, when a coupler reacts 1:2 with the diazonium salt, moderate excess of the diazonium salt maximizes yield while maintaining lower reagent costs, and that less excess is needed with mixing.

In some embodiments, the method does not include using a primary amine comprising two primary amine groups and a coupler comprising two equivalent labile hydrogens bonded to carbon. Without wishing to be bound by theory, it is believed that an undesirable polymer is formed when a primary amine comprising two primary amine groups and a coupler comprising two equivalent labile hydrogens bonded to carbon are both used.

In certain embodiments, the coupler further comprises a labile hydrogen bonded to nitrogen in an amine group.

Accordance to certain embodiments, the coupler comprises Acetoacetanalide, N-(2-Methylphenyl)-3-oxobutanamide, N-(4-Methylphenyl)-3-oxobutanamide, N-(2,4-Dimethylphenyl)-3-oxobutanamide, N-(2-Methoxyphenyl)-3-oxobutanamide, N-(4-Methoxyphenyl)-3-oxobutanamide, N-(4-Ethoxyphenyl)-3-oxobutanamide, N-(2,5-Dimethoxyphenyl)-3-oxobutanamide, 5-Acetoacetylamino benzimidazolone, N-1-Naphthyl-3-oxobutyramide, Barbituric acid, 2,4-Dihydroxyguinoline and/or 3-Methyl-1-phenyl-5-pyrazolone.

In certain embodiments, the method comprises adding a surfactant to the diazonium salt (e.g., before coupling to the coupler and/or when coupling to the coupler). In some embodiments, the surfactant comprises a water-in-oil emulsifier. In certain embodiments, the surfactant has a hydrophilic-lipophilic balance (HLB) between 4-6 (e.g., between 4-5 or between 5-6). According to some embodiments, the surfactant comprises a fatty acid ester. A non-limiting example of a fatty acid ester includes sorbitan monooleate. In some cases, the surfactant comprises n-octyl pyrrolidone.

The surfactant may be added in an appropriate amount. For example, in some cases, the method comprises adding greater than or equal to 1 wt %, greater than or equal to 3 wt %, greater than or equal to 5 wt %, greater than or equal to 7 wt %, greater than or equal to 10 wt %, greater than or equal to 12 wt %, greater than or equal to 15 wt %, or greater than or equal to 17 wt % surfactant relative to the weight of the theoretical yield of the pigment. In certain instances, the method comprises adding less than or equal to 20 wt %, less than or equal to 18 wt %, less than or equal to 15 wt %, less than or equal to 13 wt %, less than or equal to 10 wt %, less than or equal to 8 wt %, less than or equal to 5 wt %, or less than or equal to 3 wt % surfactant relative to the weight of the theoretical yield of the pigment. Combinations of these ranges are also possible (e.g., greater than or equal to 1 wt % and less than or equal to 20 wt %).

Without wishing to be bound by theory, it is believed that, in some instances, adding a surfactant to the diazonium salt (e.g., before coupling to the coupler and/or when coupling to the coupler) results in formation of a pigment with improved pigment performance.

In accordance with some embodiments, the method comprises adding an amphiphilic solvent to the diazonium salt (e.g., before coupling to the coupler and/or when coupling to the coupler). In certain cases, the amphiphilic solvent is water immiscible. For example, in some cases, less than or equal to 25% (w/w), less than or equal to 23% (w/w), less than or equal to 20% (w/w), less than or equal to 18% (w/w), less than or equal to 15% (w/w), less than or equal to 13% (w/w), less than or equal to 10% (w/w), less than or equal to 8% (w/w), less than or equal to 5% (w/w), less than or equal to 3% (w/w), or less than or equal to 1% (w/w) of the amphiphilic solvent is soluble in water. In certain instances, greater than or equal to 0% (w/w), greater than or equal to 0.05% (w/w), greater than or equal to 0.1% (w/w), greater than or equal to 0.5% (w/w), greater than or equal to 1% (w/w), greater than or equal to 3% (w/w), greater than or equal to 5% (w/w), greater than or equal to 7% (w/w), greater than or equal to 10% (w/w), greater than or equal to 12% (w/w), greater than or equal to 15% (w/w), or greater than or equal to 17% (w/w) of the amphiphilic solvent is soluble in water. Combinations of these ranges are also possible (e.g., greater than or equal to 0% (w/w) and less than or equal to 25% (w/w), greater than or equal to 0% (w/w) and less than or equal to 10% (w/w), or greater than or equal to 5% (w/w) and less than or equal to 10% (w/w)).

In some instances, the amphiphilic solvent has a chain length of greater than or equal to 6, greater than or equal to 7, greater than or equal to 8, greater than or equal to 9, greater than or equal to 10, or greater than or equal to 11. In certain cases, the amphiphilic solvent has a chain length of less than or equal to 12, less than or equal to 11, less than or equal to 10, less than or equal to 9, less than or equal to 8, or less than or equal to 7. Combinations of these ranges are also possible (e.g., greater than or equal to 6 and less than or equal to 12). In certain embodiments, the amphiphilic solvent comprises n-octyl-pyrrolidone, n-hexyl acetate, and/or n-octyl acetate.

The amphiphilic solvent may be added in an appropriate amount. For example, in some cases, the method comprises adding greater than or equal to 1 wt %, greater than or equal to 3 wt %, greater than or equal to 5 wt %, greater than or equal to 7 wt %, greater than or equal to 10 wt %, greater than or equal to 12 wt %, greater than or equal to 15 wt %, or greater than or equal to 17 wt % amphiphilic solvent relative to the weight of the theoretical yield of the pigment. In certain instances, the method comprises adding less than or equal to 20 wt %, less than or equal to 18 wt %, less than or equal to 15 wt %, less than or equal to 13 wt %, less than or equal to 10 wt %, less than or equal to 8 wt %, less than or equal to 5 wt %, or less than or equal to 3 wt % amphiphilic solvent relative to the weight of the theoretical yield of the pigment. Combinations of these ranges are also possible (e.g., greater than or equal to 1 wt % and less than or equal to 20 wt %). Without wishing to be bound by theory, it is believed that, in some instances, adding an amphiphilic solvent to the diazonium salt (e.g., before coupling to the coupler and/or when coupling to the coupler) results in formation of a pigment with improved pigment performance.

According to certain embodiments, the method comprises isolating the pigment. Non-limiting examples of isolating the pigment include washing (e.g., with water, alcohol, or a combination thereof), filtering, extracting, and/or centrifuging the pigment. For example, in some cases, the method comprises isolating the pigment, wherein isolating the pigment comprises extracting the pigment. Without wishing to be bound by theory, it is believed that, in certain instances, aqueous impurities can be extracted out in higher efficiency and with lower energy demands while pigments are easily retained in the organic layer for a simplified isolation process. In some embodiments wherein isolating the pigment comprising washing, methanol (or methanol and water combinations for pigments that have poor resistance to alcohols, for example) is used. In certain embodiments, the method comprises recycling the alcohol (e.g., methanol) from the waste stream. For example, in some cases, the method comprises recycling the alcohol (e.g., methanol) from the waste stream using distillation.

In accordance with certain embodiments, the method comprises conditioning the pigment. As used herein, “conditioning” means processing the pigment to modify its morphology. For example, in some embodiments, the method comprises conditioning the pigment in the biphasic mixture. In certain cases, the method comprises conditioning the pigment prior to isolating the pigment. In some instances, conditioning the pigment comprises heating the reaction mixture (e.g., the biphasic mixture). For example, in some cases conditioning the pigment comprises heating the reaction mixture to near the boiling point of the organic solvent (e.g., in the biphasic mixture). Without wishing to be bound by theory, it is believed that pigments may have improved properties from conditioning at higher temperatures to transform their crystal structure to different polymorphs, as higher temperature polymorphs may have improved pigment performance and stability, in accordance with certain embodiments.

In some embodiments, the method comprises recycling at least a portion (e.g., at least 10%, at least 25%, at least 50%, at least 75%, at least 90%, or all) of the organic solvent. For example, in certain cases, the method comprises recycling at least a portion of the organic solvent by distilling at least a portion (e.g., at least 10%, at least 25%, at least 50%, at least 75%, at least 90%, or all) of the organic solvent. For example, in some instances, the method comprises recycling at least a portion of the organic solvent by distilling at least a portion (e.g., at least 10%, at least 25%, at least 50%, at least 75%, at least 90%, or all) of the organic solvent when conditioning the pigment.

In certain embodiments, the pigment comprises an azo pigment, a monoazo pigment, a bisazo pigment, and/or a pigment lake. For example, in certain cases, the pigment comprises an azo pigment. Non-limiting examples of azo pigments include red azo pigments, orange azo pigments, yellow azo pigments, violet azo pigments, and/or brown azo pigments. For example, in some instances, the azo pigment comprises a yellow azo pigment.

In certain instances, the pigment is non-halogenated. Without wishing to be bound by theory, it is believed that non-halogenated pigments may be less toxic and/or carcinogenic, in some cases.

In some embodiments, the pigment comprises a pigment lake (or a lake pigment). In certain embodiments where the pigment comprises a pigment lake, the method comprises removing an aqueous layer (e.g., from the biphasic mixture) after reacting the diazonium salt with the coupler. In some cases where the pigment comprises a pigment lake, the method comprises adding a solution comprising a divalent or trivalent metal species (e.g., replacing the removed aqueous layer with the solution comprising the divalent or trivalent metal species). The divalent or trivalent metal species are calcium, nickel, strontium, aluminum, and/or magnesium, in accordance with some embodiments. In certain instances where the pigment comprises a pigment lake, forming the pigment comprises precipitating the pigment from an organic layer. For example, in some instances, forming the pigment comprises precipitating the pigment from an organic layer upon mixing thoroughly. As another example, in certain cases, forming the pigment comprises precipitating the pigment from an organic layer upon mixing thoroughly while heating.

In certain embodiments, the pigment lake comprises a calcium pigment lake. In some embodiments where the pigment lake comprises a calcium pigment lake, the method comprises dissolving at least a portion (e.g., at least 10%, at least 25%, at least 50%, at least 75%, at least 90%, or all) of the coupler in a calcium bicarbonate solution. For example, in some cases where the pigment lake comprises a calcium pigment lake, the method comprises dissolving at least a portion (e.g., at least 10%, at least 25%, at least 50%, at least 75%, at least 90%, or all) of the coupler in a calcium bicarbonate solution prior to reacting the diazonium salt with the coupler. In some embodiments, dissolving at least a portion of the coupler in a calcium bicarbonate solution prior to reacting the diazonium salt with the coupler results in the direct formation of a calcium pigment lake.

As used herein, a pigment is a solid colorant which is dispersed into a material while in solid form. For example, a pigment is a solid that is insoluble in a material (or solvent added thereto) but still adds color to that material without being dissolved into the material. In contrast, dyes are colored substances that add color to a material when dissolved into the material.

The pigment may have a suitable average particle size. According to some embodiments, the pigment has an average particle size of greater than or equal to 50 nanometers, greater than or equal to 100 nanometers, greater than or equal to 200 nanometers, greater than or equal to 300 nanometers, greater than or equal to 400 nanometers, greater than or equal to 500 nanometers, greater than or equal to 600 nanometers, greater than or equal to 700 nanometers, greater than or equal to 800 nanometers, or greater than or equal to 900 nanometers. In certain cases, the pigment has an average particle size of less than or equal to 1000 nanometers, less than or equal to 900 nanometers, less than or equal to 800 nanometers, less than or equal to 700 nanometers, less than or equal to 600 nanometers, less than or equal to 500 nanometers, less than or equal to 400 nanometers, less than or equal to 300 nanometers, less than or equal to 200 nanometers, or less than or equal to 100 nanometers. Combinations of these ranges are also possible (e.g., greater than or equal to 50 nanometers and less than or equal to 1000 nanometers).

In some embodiments, the method (e.g., including any conditioning steps) does not include any chlorinated reagents or substrates. For example, in certain cases, the method (e.g., including any conditioning steps) does not include any halogenated reagents or substrates. Without wishing to be bound by theory, it is believed that not including any chlorinated reagents or substrates and/or not including any halogenated reagents or substrates results in reduced toxic and/or carcinogenic byproducts, in some cases, as using hydrochloric acid, chlorinated solvents, and/or other halogenated reagents or substrates may generate toxic and/or carcinogenic byproducts, such as polychlorinated biphenyl species.

It should be understood that any of the method steps disclosed herein may each independently include mixing, in some embodiments.

Certain aspects are related to pigments (e.g., any pigment disclosed herein). In some cases, the pigment is formed by a method (e.g., any method disclosed herein).

In certain embodiments, the pigment comprises one or more of:

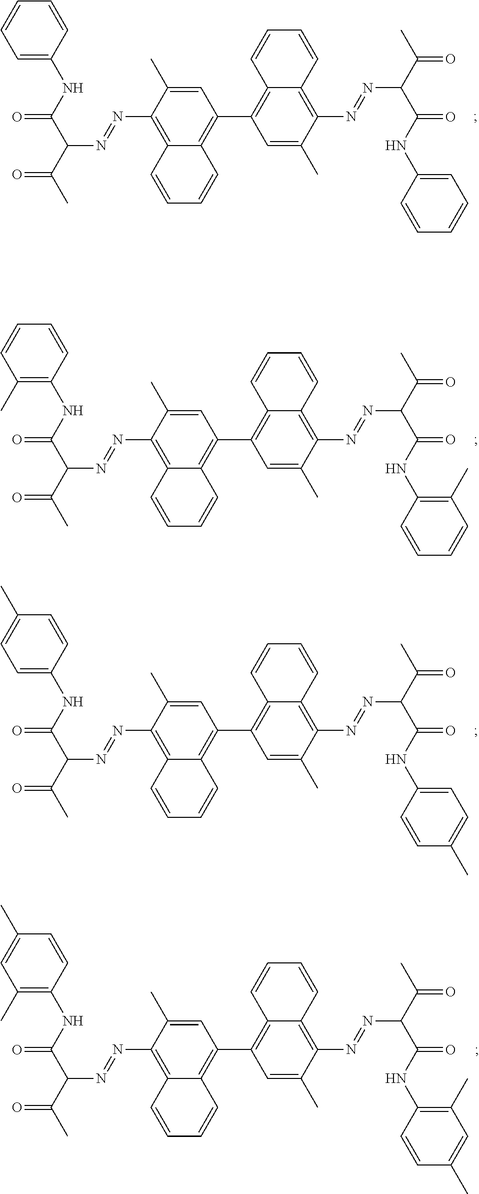 [SMILES: CC(=O)C(/N=N/c1c(C)cc(-c2cc(C)c(/N=N/C(C(C)=O)C(=O)Nc3ccccc3)c3ccccc23)c2ccccc12)C(=O)Nc1ccccc1 ; CC(=O)C(/N=N/c1c(C)cc(-c2cc(C)c(/N=N/C(C(C)=O)C(=O)Nc3ccccc3C)c3ccccc23)c2ccccc12)C(=O)Nc1ccccc1C ; CC(=O)C(/N=N/c1c(C)cc(-c2cc(C)c(/N=N/C(C(C)=O)C(=O)Nc3ccc(C)cc3)c3ccccc23)c2ccccc12)C(=O)Nc1ccc(C)cc1 ; CC(=O)C(/N=N/c1c(C)cc(-c2cc(C)c(/N=N/C(C(C)=O)C(=O)Nc3ccc(C)cc3C)c3ccccc23)c2ccccc12)C(=O)Nc1ccc(C)cc1C]

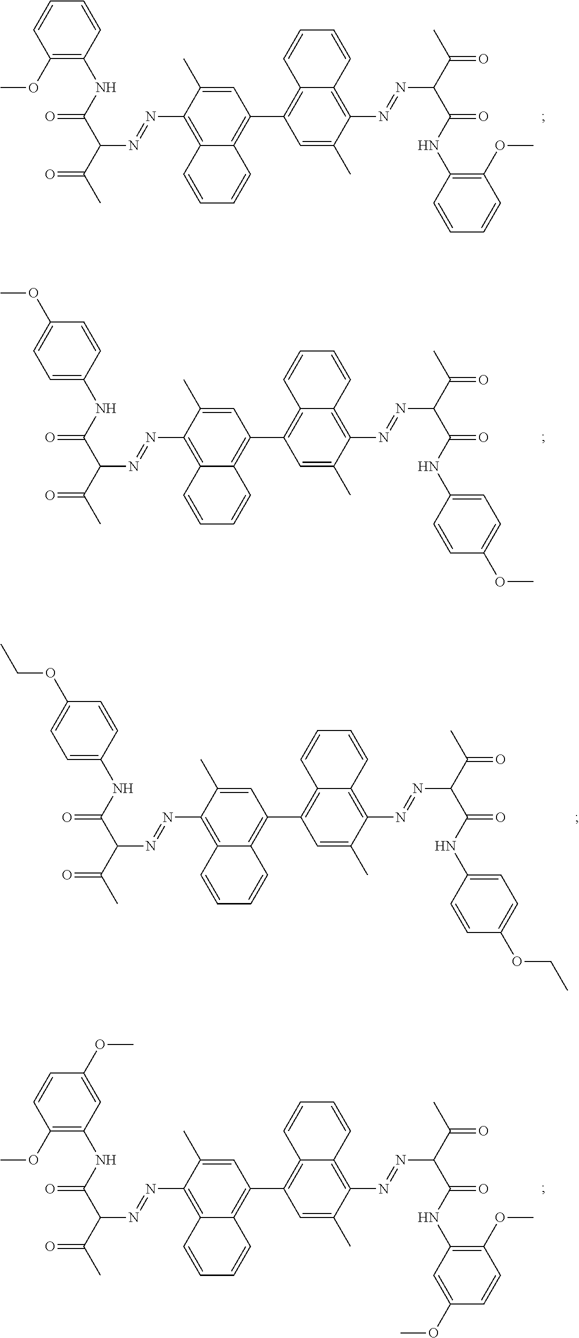

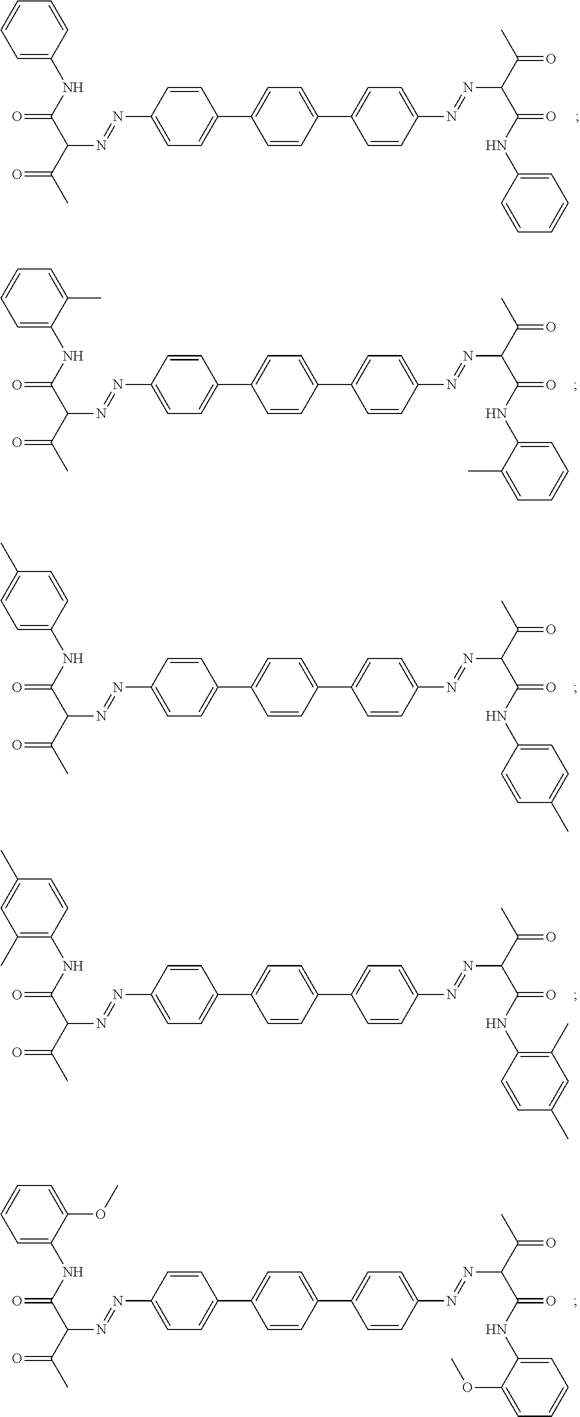 [SMILES: CC(=O)C(/N=N/c1ccc(-c2ccc(-c3ccc(/N=N/C(C(C)=O)C(=O)Nc4ccccc4)cc3)cc2)cc1)C(=O)Nc1ccccc1 ; CC(=O)C(/N=N/c1ccc(-c2ccc(-c3ccc(/N=N/C(C(C)=O)C(=O)Nc4ccccc4C)cc3)cc2)cc1)C(=O)Nc1ccccc1C ; CC(=O)C(/N=N/c1ccc(-c2ccc(-c3ccc(/N=N/C(C(C)=O)C(=O)Nc4ccc(C)cc4)cc3)cc2)cc1)C(=O)Nc1ccc(C)cc1 ; CC(=O)C(/N=N/c1ccc(-c2ccc(-c3ccc(/N=N/C(C(C)=O)C(=O)Nc4ccc(C)cc4C)cc3)cc2)cc1)C(=O)Nc1ccc(C)cc1C ; COc1ccccc1NC(=O)C(/N=N/c1ccc(-c2ccc(-c3ccc(/N=N/C(C(C)=O)C(=O)Nc4ccccc4OC)cc3)cc2)cc1)C(C)=O]

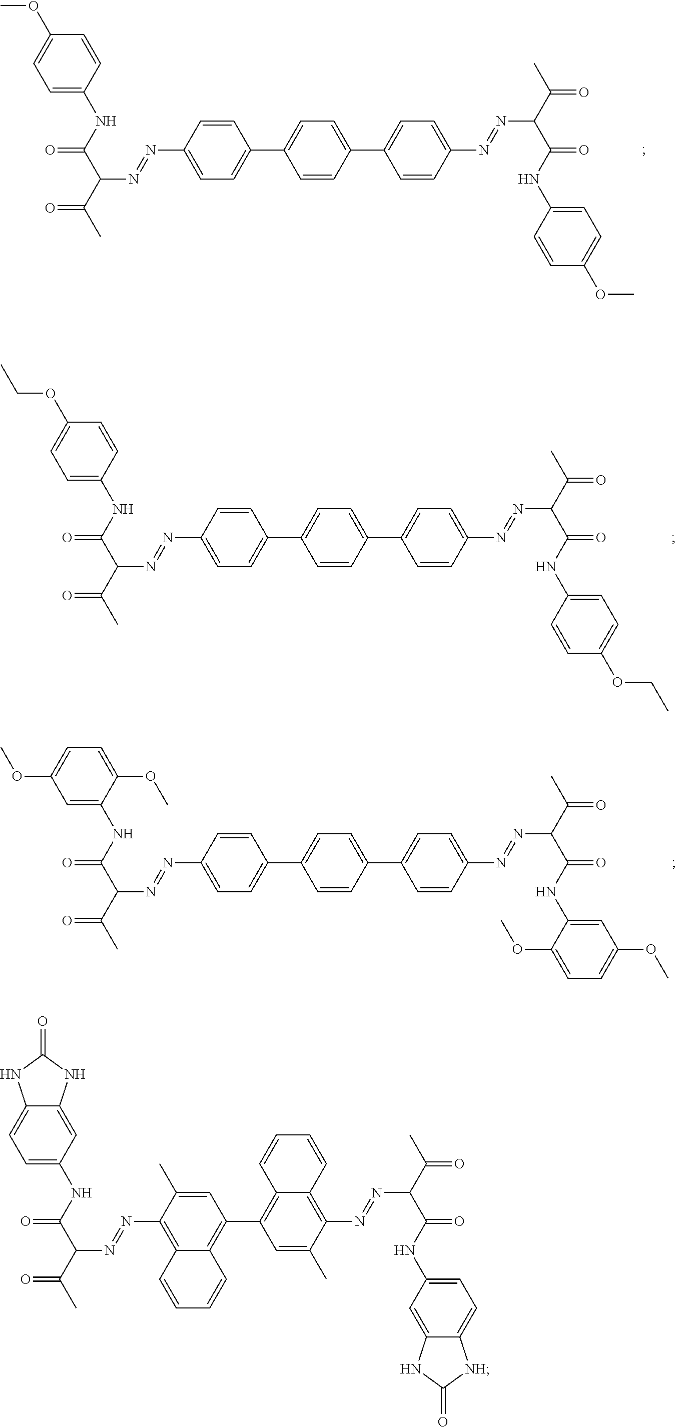

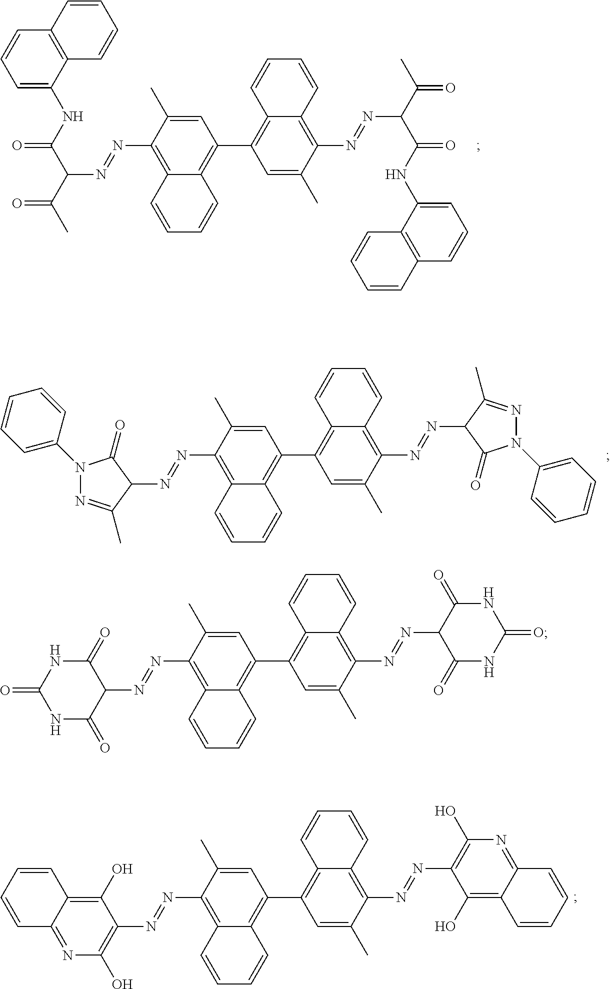 [SMILES: CC(=O)C(/N=N/c1c(C)cc(-c2cc(C)c(/N=N/C(C(C)=O)C(=O)Nc3cccc4ccccc34)c3ccccc23)c2ccccc12)C(=O)Nc1cccc2ccccc12 ; CC1=NN(c2ccccc2)C(=O)C1/N=N/c1c(C)cc(-c2cc(C)c(/N=N/C3C(=O)N(c4ccccc4)N=C3C)c3ccccc23)c2ccccc12 ; Cc1cc(-c2cc(C)c(/N=N/C3C(=O)NC(=O)NC3=O)c3ccccc23)c2ccccc2c1/N=N/C1C(=O)NC(=O)NC1=O ; Cc1cc(-c2cc(C)c(/N=N/c3c(O)nc4ccccc4c3O)c3ccccc23)c2ccccc2c1/N=N/c1c(O)nc2ccccc2c1O]

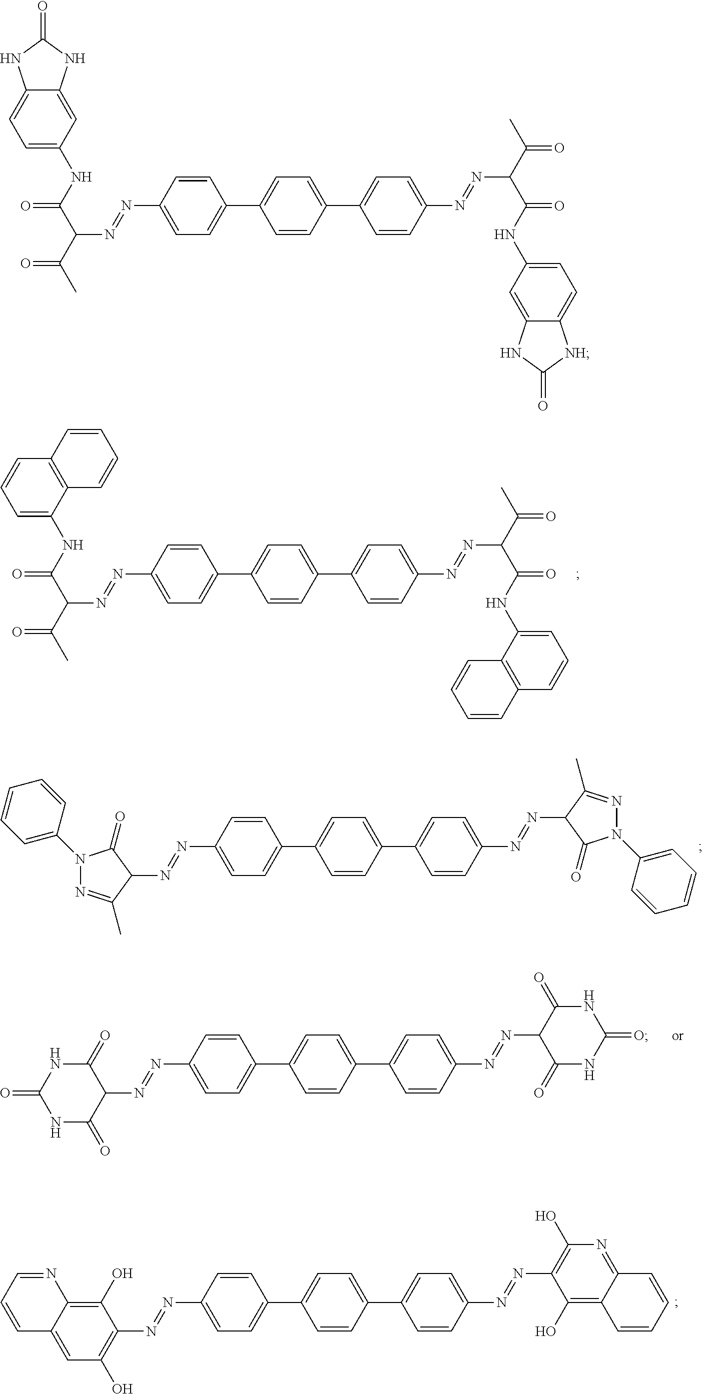 [SMILES: CC(=O)C(/N=N/c1ccc(-c2ccc(-c3ccc(/N=N/C(C(C)=O)C(=O)Nc4ccc5[nH]c(=O)[nH]c5c4)cc3)cc2)cc1)C(=O)Nc1ccc2[nH]c(=O)[nH]c2c1 ; CC(=O)C(/N=N/c1ccc(-c2ccc(-c3ccc(/N=N/C(C(C)=O)C(=O)Nc4cccc5ccccc45)cc3)cc2)cc1)C(=O)Nc1cccc2ccccc12 ; CC1=NN(c2ccccc2)C(=O)C1/N=N/c1ccc(-c2ccc(-c3ccc(/N=N/C4C(=O)N(c5ccccc5)N=C4C)cc3)cc2)cc1 ; O=C1NC(=O)C(/N=N/c2ccc(-c3ccc(-c4ccc(/N=N/C5C(=O)NC(=O)NC5=O)cc4)cc3)cc2)C(=O)N1 ; Oc1cc2cccnc2c(O)c1/N=N/c1ccc(-c2ccc(-c3ccc(/N=N/c4c(O)nc5ccccc5c4O)cc3)cc2)cc1]

or a tautomer or polymorph thereof.

In some cases, the pigment comprises:

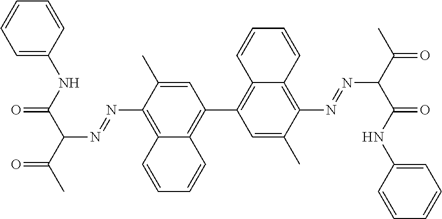 [SMILES: CC(=O)C(/N=N/c1c(C)cc(-c2cc(C)c(/N=N/C(C(C)=O)C(=O)Nc3ccccc3)c3ccccc23)c2ccccc12)C(=O)Nc1ccccc1]

N-Phenyl2-[(E)-{3,3′-dimethyl-4′-[(E)-1-(N-phenylcarbamoyl)-2-oxopropylazo]-1,1′-binaphthyl-4-yl}azo]acetoacetamide
O═C(NC(═CC═C1)C═C1)C(/N═N/C(C(C)═CC1C(C═C(C)C2/N═N/C(C(═O)NC(C═CC3)=CC═3)C(═O)C)═C(C═CC3)C═2C=3)=C(C═CC2)C═1C=2)C(═O)C;

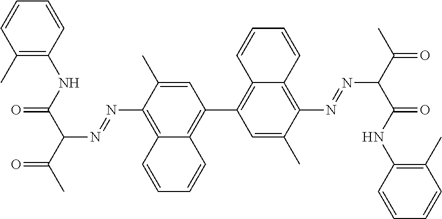 [SMILES: CC(=O)C(/N=N/c1c(C)cc(-c2cc(C)c(/N=N/C(C(C)=O)C(=O)Nc3ccccc3C)c3ccccc23)c2ccccc12)C(=O)Nc1ccccc1C]

N-o-Tolyl2-[(E)-{3,3′-dimethyl-4′-[(E)-1-(N-o-tolylcarbamoyl)-2-oxopropylazo]-1,1′-binaphthyl-4-yl}azo]acetoacetamide
O═C(NC(C═C1)=C(C)C═C1)C(/N═N/C(C(C)═CC1C(C═C(C)C2/N═N/C(C(C)═O)C(═O)NC(C═CC3)=C(C)C=3)=C(C=2C═C2)C═C2)=C(C=1C═C1)C═C1)C(═O)C;

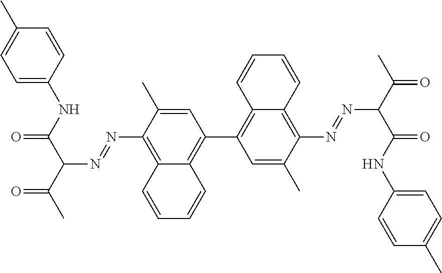 [SMILES: CC(=O)C(/N=N/c1c(C)cc(-c2cc(C)c(/N=N/C(C(C)=O)C(=O)Nc3ccc(C)cc3)c3ccccc23)c2ccccc12)C(=O)Nc1ccc(C)cc1]

N-p-Tolyl2-[(E)-{3,3′-dimethyl-4′-[(E)-1-(N-p-tolylcarbamoyl)-2-oxopropylazo]-1,1′-binaphthyl-4-yl}azo]acetoacetamide
O═C(NC(C═CC1C)═CC=1)C(/N═N/C(C(C)═CC1C(C═C2C)═C(C═CC3)C(C=3)=C2/N═N/C(C(═O)NC(C═CC2C)═CC=2)C(═O)C)═C(C=1C═C1)C═C1)C(═O)C;

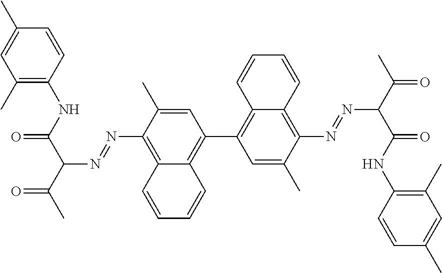 [SMILES: CC(=O)C(/N=N/c1c(C)cc(-c2cc(C)c(/N=N/C(C(C)=O)C(=O)Nc3ccc(C)cc3C)c3ccccc23)c2ccccc12)C(=O)Nc1ccc(C)cc1C]

N-2,4-Xylyl2-[(E)-{3,3′-dimethyl-4′-[(E)-1-(N-2,4-xylylcarbamoyl)-2-oxopropylazo]-1,1′-binaphthyl-4-yl}azo]acetoacetamide
O═C(C)C(/N═N/C(═C(C═C1)C(C═C1)=C1C(C═C2C)═C(C═CC3)C(C═3)=C2/N═N/C(C(C)═O)C(═O)NC(C═CC2C)═C(C=2)C)C(C)═C1)C(═O)NC(C═C1)=C(C)C═C1C;

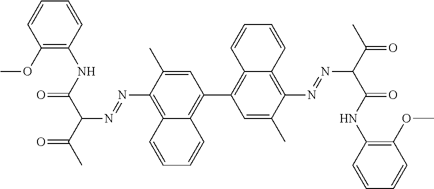

N-o-Methoxyphenyl2-[(E)-{3,3′-dimethyl-4′-[(E)-1-(N-o-methoxyphenylcarbamoyl)-2-oxopropylazo]-1,1′-binaphthyl-4-yl}azo]acetoacetamide
O═C(C)C(/N═N/C(C(C)═CC1C(C═C(C)C2/N═N/C(C(═O)C)C(═O)NC(C═CC3)=C(C=3)OC)═C(C=2C═C2)C═C2)=C(C=1C═C1)C═C1)C(═O)NC(C═C1)=C(OC)C═C1;

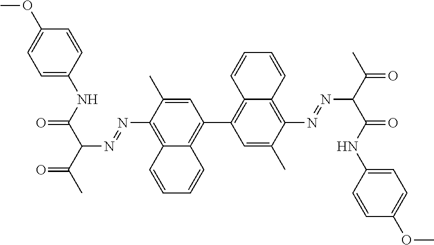

N-p-Methoxyphenyl2-[(E)-{3,3′-dimethyl-4′-[(E)-1-(N-p-methoxyphenylcarbamoyl)-2-oxopropylazo]-1,1′-binaphthyl-4-yl}azo]acetoacetamide
O═C(C)C(/N═N/C(═C(C═C1)C(C═C1)=C1C(C═C(C)C2/N═N/C(C(═O)C)C(═O) NC(C═CC3OC)═CC=3)=C(C=2C═C2)C═C2)C(C)═C1)C(═O)NC(C═CC1OC)═CC=1;

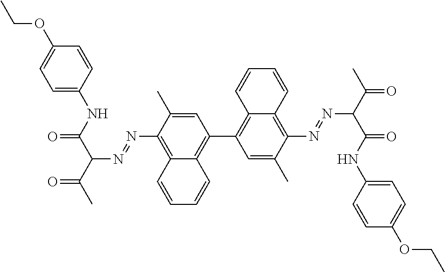 [SMILES: CCOc1ccc(NC(=O)C(/N=N/c2c(C)cc(-c3cc(C)c(/N=N/C(C(C)=O)C(=O)Nc4ccc(OCC)cc4)c4ccccc34)c3ccccc23)C(C)=O)cc1]

N-p-Ethoxyphenyl2-[(E)-{3,3′-dimethyl-4′-[(E)-1-(N-p-ethoxyphenylcarbamoyl)-2-oxopropylazo]-1,1′-binaphthyl-4-yl}azo]acetoacetamide
O═C(C)C(/N═N/C(═C(C═CC1)C(C=1)=C1C(C═C2C)═C(C═C3)C(C═C3)=C2/N═N/C(C(═O)NC(═CC═C2OCC)C═C2)C(C)═O)C(═C1)C)C(═O)NC(C═CC1OCC)═CC=1;

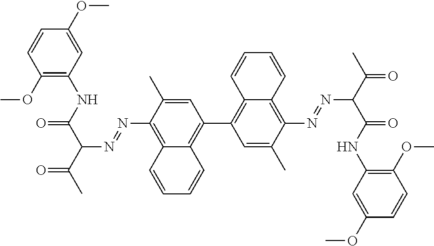

N-2,5-Dimethoxyphenyl2-[(E)-{3,3′-dimethyl-4′-[(E)-1-(N-2,5-dimethoxyphenylcarbamoyl)-2-oxopropylazo]-1,1′-binaphthyl-4-yl}azo]acetoacetamide
O═C(C)C(/N═N/C(C(C)═CC1C(C═C(C)C2/N═N/C(C(═O)NC(C═C3OC)═C(C═C3)OC)C(C)=O)═C(C=2C═C2)C═C2)=C(C═CC2)C=1C=2)C(═O)NC(C═C(C1)OC)═C(C=1)OC;

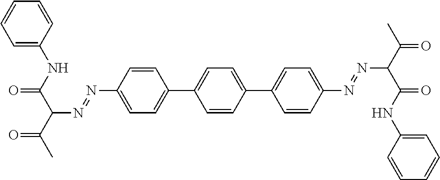 [SMILES: CC(=O)C(/N=N/c1ccc(-c2ccc(-c3ccc(/N=N/C(C(C)=O)C(=O)Nc4ccccc4)cc3)cc2)cc1)C(=O)Nc1ccccc1]

N-Phenyl3-oxo-2-[(E)-{4″-[(E)-1-(N-phenylcarbamoyl)-2-oxopropylazo]-p-terphenyl-4-yl}azo]butyramide
O═C(NC(═CC═C1)C═C1)C(/N═N/C(═CC═CIC(═CC═C2C(C═CC3/N═N/C(C(═O)NC(═CC═C4)C═C4)C(C)=O)═CC=3)C═C2)C═C1)C(═O)C;

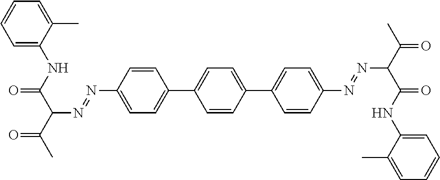 [SMILES: CC(=O)C(/N=N/c1ccc(-c2ccc(-c3ccc(/N=N/C(C(C)=O)C(=O)Nc4ccccc4C)cc3)cc2)cc1)C(=O)Nc1ccccc1C]

N-o-Tolyl3-oxo-2-[(E)-{4″-[(E)-1-(N-o-tolylcarbamoyl)-2-oxopropylazo]-p-terphenyl-4-yl}azo]butyramide
O═C(C)C(/N═N/C(═CC═C1C(═CC═C2C(═CC═C3/N═N/C(C(═O)C)C(═O)NC(═CC4)C(C)═CC=4)C═C3)C═C2)C═C1)C(═O)NC(═CC1)C(C)═CC=1;

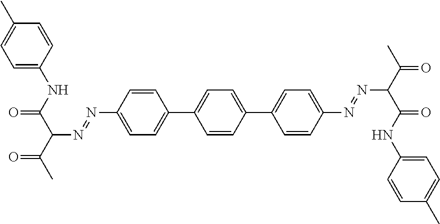 [SMILES: CC(=O)C(/N=N/c1ccc(-c2ccc(-c3ccc(/N=N/C(C(C)=O)C(=O)Nc4ccc(C)cc4)cc3)cc2)cc1)C(=O)Nc1ccc(C)cc1]

N-p-Tolyl3-oxo-2-[(E)-{4″-[(E)-1-(N-p-tolylcarbamoyl)-2-oxopropylazo]-p-terphenyl-4-yl}azo]butyramide
O═C(C)C(/N═N/C(═CC═C1C(C═CC2C(═CC═C3/N═N/C(C(═O)C)C(═O)NC(═CC═C4C)C═C4)C═C3)=CC=2)C═C1)C(═O)NC(═CC═C1C)C═C1;

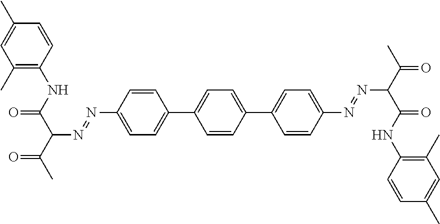 [SMILES: CC(=O)C(/N=N/c1ccc(-c2ccc(-c3ccc(/N=N/C(C(C)=O)C(=O)Nc4ccc(C)cc4C)cc3)cc2)cc1)C(=O)Nc1ccc(C)cc1C]

N-2,4-Xylyl3-oxo-2-[(E)-{4″-[(E)-1-(N-2,4-xylylcarbamoyl)-2-oxopropylazo]-p-terphenyl-4-yl}azo]butyramide
O═C(C)C(/N═N/C(═CC═C1C(═CC═C2C(═CC═C3/N═N/C(C(═O)C)C(═O)NC(C═CC4C)═C(C=4)C)C═C3)C═C2)C═C1)C(═O)NC(C═CC1C)═C(C=1)C;

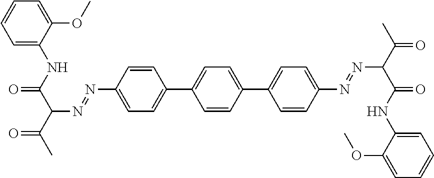 [SMILES: COc1ccccc1NC(=O)C(/N=N/c1ccc(-c2ccc(-c3ccc(/N=N/C(C(C)=O)C(=O)Nc4ccccc4OC)cc3)cc2)cc1)C(C)=O]

N-o-Methoxyphenyl3-oxo-2-[(E)-{4″-[(E)-1-(N-o-methoxyphenylcarbamoyl)-2-oxopropylazo]-p-terphenyl-4-yl}azo]butyramide
O═C(C)C(/N═N/C(═CC═C1C(═CC═C2C(C═CC3/N═N/C(C(═O)NC(═CC4)C(═CC=4)OC)C(═O)C)═CC=3)C═C2)C═C1)C(═O)NC(═CC1)C(OC)═CC=1;

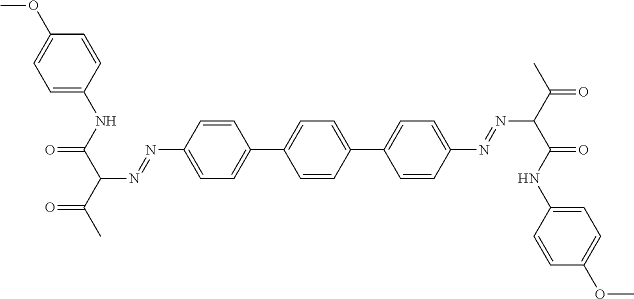

N-p-Methoxyphenyl3-oxo-2-[(E)-{4″-[(E)-1-(N-p-methoxyphenylcarbamoyl)-2-oxopropylazo]-p-terphenyl-4-yl}azo]butyramide
O═C(NC(C═CC1OC)═CC=1)C(/N═N/C(C═CC1C(C═CC2C(═CC═C3/N═N/C(C(═O)NC(C═CC4OC)═CC=4)C(C)═O)C═C3)=CC=2)=CC=1)C(C)═O;

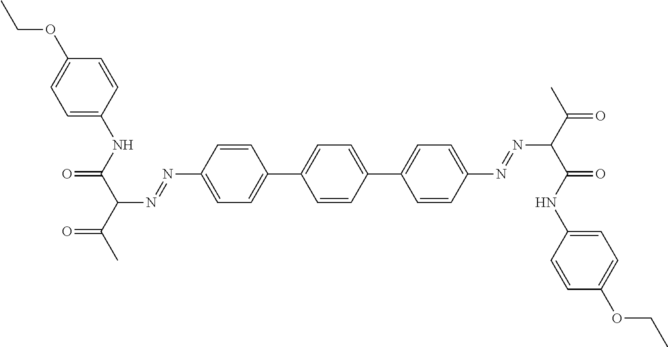 [SMILES: CCOc1ccc(NC(=O)C(/N=N/c2ccc(-c3ccc(-c4ccc(/N=N/C(C(C)=O)C(=O)Nc5ccc(OCC)cc5)cc4)cc3)cc2)C(C)=O)cc1]

N-p-Ethoxyphenyl3-oxo-2-[(E)-{4″-[(E)-1-(N-p-ethoxyphenylcarbamoyl)-2-oxopropylazo]-p-terphenyl-4-yl}azo]butyramide
O═C(C)C(/N═N/C(═CC═C1C(═CC═C2C(C═CC3/N═N/C(C(C)═O)C(═O)NC(═CC═C4OCC)C═C4)=CC=3)C═C2)C═C1)C(═O)NC(═CC═C1OCC)C═C1;

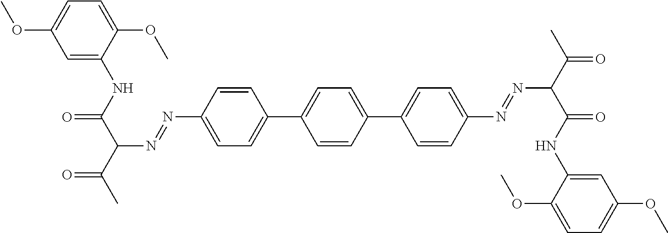

N-2,5-Dimethoxyphenyl3-oxo-2-[(E)-{4″-[(E)-1-(N-2,5-dimethoxyphenylcarbamoyl)-2-oxopropylazo]-p-terphenyl-4-yl}azo]butyramide
O═C(C)C(/N═N/C(C═CC1C(C═CC2C(═CC═C3/N═N/C(C(═O)C)C(═O)NC(═C C4OC)C(═CC=4)OC)C═C3)=CC=2)=CC=1)C(═O)NC(═CC1OC)C(OC)═CC=1;

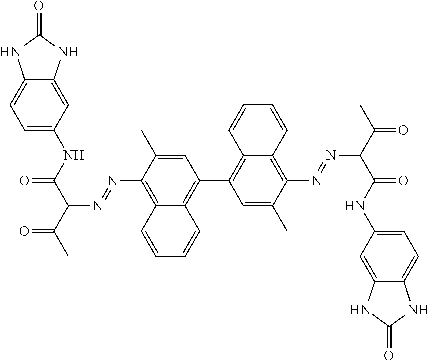 [SMILES: CC(=O)C(/N=N/c1c(C)cc(-c2cc(C)c(/N=N/C(C(C)=O)C(=O)Nc3ccc4[nH]c(=O)[nH]c4c3)c3ccccc23)c2ccccc12)C(=O)Nc1ccc2[nH]c(=O)[nH]c2c1]

N-(2-Oxo-2,3-dihydro-1H-1,3-benzimidazol-5-yl) 2-[(E)-{3,3′-dimethyl-4′-[(E)-1-(N-2-oxo-2,3-dihydro-1H-1,3-benzimidazol-5-ylcarbamoyl)-2-oxopropylazo]-1,1′-binaphthyl-4-yl}azo]acetoacetamide
O═C(NC(═CC═C1N2)C═C1NC2=O)C(/N═N/C(C(C)═CC1C(C═C(C)C2/N═N/C(C(C)═O)C(═O)NC(═CC═C3N4)C═C3NC4=O)=C(C═CC3)C=2C=3)=C(C=1C═C1)C═C1)C(═O)C;

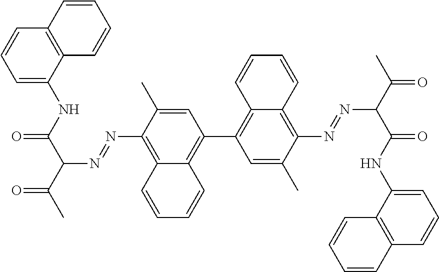 [SMILES: CC(=O)C(/N=N/c1c(C)cc(-c2cc(C)c(/N=N/C(C(C)=O)C(=O)Nc3cccc4ccccc34)c3ccccc23)c2ccccc12)C(=O)Nc1cccc2ccccc12]

N-1-Naphthyl2-[(E)-{3,3′-dimethyl-4′-[(E)-1-(N-1-naphthylcarbamoyl)-2-oxopropylazo]-1,1′-binaphthyl-4-yl}azo]acetoacetamide
O═C(C)C(/N═N/C(C(C)═CC1C(C═C2C)═C(C═C3)C(═C2/N═N/C(C(C)═O)C(═O)NC(═CC═C2)C(═C2C═C2)C═C2)C═C3)=C(C=1C═C1)C═C1)C(═O)NC(═CC═C1)C(═C1C═C1)C═C1;

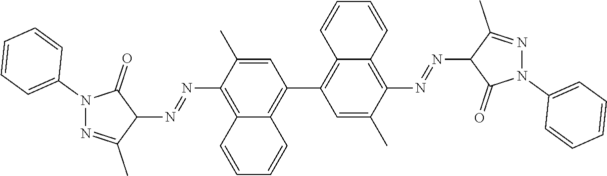 [SMILES: CC1=NN(c2ccccc2)C(=O)C1/N=N/c1c(C)cc(-c2cc(C)c(/N=N/C3C(=O)N(c4ccccc4)N=C3C)c3ccccc23)c2ccccc12]

4-[(E)-{3,3′-Dimethyl-4′-[(E)-3-methyl-5-oxo-1-phenyl-2-pyrazolin-4-ylazo]-1,1′-binaphthyl-4-yl}azo]-3-methyl-1-phenyl-2-pyrazolin-5-one
O═C(N(N1)C(C═CC2)=CC=2)C(/N═N/C(═C(C═CC2)C(C=2)=C2C(C═C3C)═C(C═C4)C(═C3/N═N/C(C(═N3)C)C(═O)N3C(═CC═C3)C═C3)C═C4)C(C)═C2)C=1C;

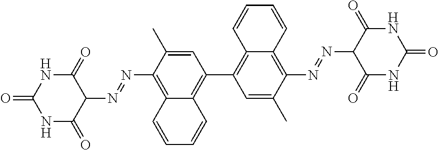 [SMILES: Cc1cc(-c2cc(C)c(/N=N/C3C(=O)NC(=O)NC3=O)c3ccccc23)c2ccccc2c1/N=N/C1C(=O)NC(=O)NC1=O]

5-[(E)-{3,3′-Dimethyl-4′-[(E)-2,4,6-trioxohexahydro-5-pyrimidinylazo]-1,1′-binaphthyl-4-yl}azo]-2,4,6(1H,3H,5H)-pyrimidinetrione
O═C(NC(N1)=O)C(/N═N/C(C(C)═CC2C(C═C(C)C3/N═N/C(C(═O)NC4=O)C(N4)=O)═C(C=3C═C3)C═C3)=C(C═CC3)C=2C=3)C1=O;

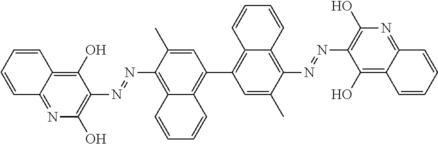 [SMILES: Cc1cc(-c2cc(C)c(/N=N/c3c(O)nc4ccccc4c3O)c3ccccc23)c2ccccc2c1/N=N/c1c(O)nc2ccccc2c1O]

3-[(E)-{4′-[(E)-2,4-Dihydroxy-3-quinolylazo]-3,3′-dimethyl-1,1′-binaphthyl-4-yl}azo]-2,4-quinolinediol
OC(N═C1C═C2)=C(/N═N/C(C(C)═CC3C(C═C4C)═C(C═C5)C(═C4/N═N/C(C(O)═C(C4C═C6)C═C6)=C(O)N=4)C═C5)=C(C═CC4)C=3C=4)C(O)=C1C═C2;

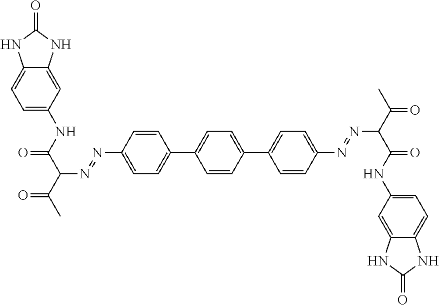 [SMILES: CC(=O)C(/N=N/c1ccc(-c2ccc(-c3ccc(/N=N/C(C(C)=O)C(=O)Nc4ccc5[nH]c(=O)[nH]c5c4)cc3)cc2)cc1)C(=O)Nc1ccc2[nH]c(=O)[nH]c2c1]

N-(2-Oxo-2,3-dihydro-1H-1,3-benzimidazol-5-yl)3-oxo-2-[(E)-{4″-[(E)-1-(N-2-oxo-2,3-dihydro-1H-1,3-benzimidazol-5-ylcarbamoyl)-2-oxopropylazo]-p-terphenyl-4-yl}azo]butyramide
O═C(NC(═CC═C1N2)C═C1NC2=O)C(/N═N/C(═CC═C1C(C═CC2C(C═CC3/N═N/C(C(═O)NC(C═C(C4N5)NC5=O)═CC=4)C(═O)C)═CC=3)=CC=2)C═C1)C(═O)C;

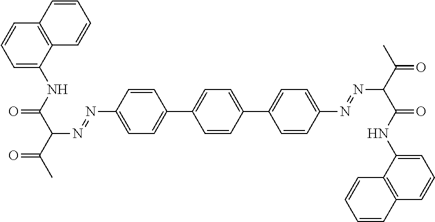 [SMILES: CC(=O)C(/N=N/c1ccc(-c2ccc(-c3ccc(/N=N/C(C(C)=O)C(=O)Nc4cccc5ccccc45)cc3)cc2)cc1)C(=O)Nc1cccc2ccccc12]

N-1-Naphthyl3-oxo-2-[(E)-{4″-[(E)-1-(N-1-naphthylcarbamoyl)-2-oxopropylazo]-p-terphenyl-4-yl}azo]butyramide
O═C(C)C(/N═N/C(═CC═C1C(C═CC2C(═CC═C3/N═N/C(C(═O)NC(═CC═C4)C(═C4C═C4)C═C4)C(═O)C)C═C3)=CC=2)C═C1)C(═O)NC(═CC1)C(C═C2)=C(C=1)C═C2;

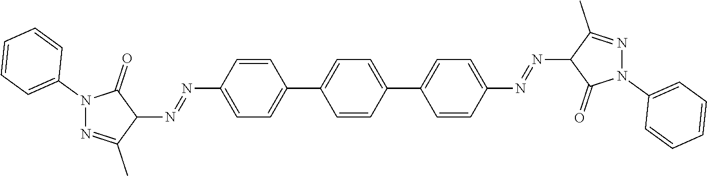 [SMILES: CC1=NN(c2ccccc2)C(=O)C1/N=N/c1ccc(-c2ccc(-c3ccc(/N=N/C4C(=O)N(c5ccccc5)N=C4C)cc3)cc2)cc1]

3-Methyl-4-[(E)-{4″-[(E)-3-methyl-5-oxo-1-phenyl-2-pyrazolin-4-ylazo]-p-terphenyl-4-yl}azo]-1-phenyl-2-pyrazolin-5-one
O═C(C(C1C)/N═N/C(C═CC2C(C═CC3C(═CC═C4/N═N/C(C(C)═N5)C(═O)N5C(C═CC5)=CC=5)C═C4)=CC=3)=CC=2)N(N=1)C(C═CC1)=CC=1;

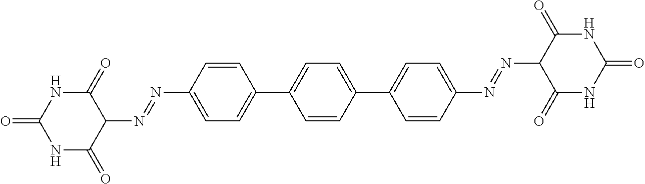 [SMILES: O=C1NC(=O)C(/N=N/c2ccc(-c3ccc(-c4ccc(/N=N/C5C(=O)NC(=O)NC5=O)cc4)cc3)cc2)C(=O)N1]

5-[(E)-{4″-[(E)-2,4,6-Trioxohexahydro-5-pyrimidinylazo]-p-terphenyl-4-yl}azo]-2,4,6(1H,3H,5H)-pyrimidinetrione
O═C(NC(N1)=O)C(C1=O)/N═N/C(═CC═C1C(C═CC2C(C═CC3/N═N/C(C(═O) NC4=O)C(N4)=O)═CC=3)=CC=2)C═C1;

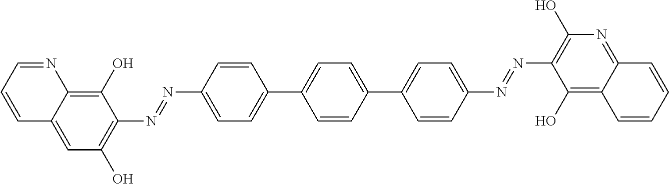 [SMILES: Oc1cc2cccnc2c(O)c1/N=N/c1ccc(-c2ccc(-c3ccc(/N=N/c4c(O)nc5ccccc5c4O)cc3)cc2)cc1]

3-[(E)-{4″-[(E)-6,8-Dihydroxy-7-quinolylazo]-p-terphenyl-4-yl}azo]-2,4-quinolinediol
OC(C(/N═N/C(C═CC1C(C═CC2C(═CC═C3/N═N/C(C(O)═C(C═CC4)C5C=4)=C(N=5)O)C═C3)=CC=2)=CC=1)=C(O)C1)=C(C=1C═C1)N═C1;
or a tautomer or polymorph thereof.

In some cases, the tautomer is an azo-hydrazone tautomer. For example, the structures shown herein are generally shown in the azo tautomer form but may also exist in the hydrazone form (e.g., in crystalline pigments and/or in pigment solutions). In certain cases, the pigment is present in the azo tautomer form. In some instance, the pigment is present in the hydrazone tautomer form. According to certain embodiments, a portion of the pigment is present in the azo tautomer form and a portion of the pigment is present in the hydrazone tautomer form. For the sake of illustration, an example of azo-hydrazone tautomerism is shown as follows:

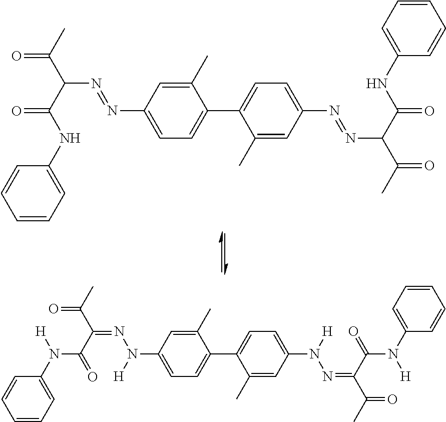 [SMILES: CC(=O)C(/N=N/c1ccc(-c2ccc(/N=N/C(C(C)=O)C(=O)Nc3ccccc3)cc2C)c(C)c1)C(=O)Nc1ccccc1 ; CC(=O)/C(=N/Nc1ccc(-c2ccc(N/N=C(/C(C)=O)C(=O)Nc3ccccc3)cc2C)c(C)c1)C(=O)Nc1ccccc1]

The following examples are intended to illustrate certain embodiments of the present invention, but do not exemplify the full scope of the invention.

### EXAMPLE 1

This example describes various methods of forming pigments.

### Method A

A diazonium salt was generated when 0.2 mmol of a primary amine was reacted with >0.2 mmol of a nitrite ester in the presence of >0.2 mmol of an acid. Preferred results were obtained when the nitrite ester and acid were used in excess of 0.22 mmol and 0.21 mmol, respectively, in good mixing conditions. The diazonium salt was then reacted with >0.2 mmol of a coupler, with preferred results obtained with 0.21 mmol coupler in good mixing conditions. An example of Method A is shown below:

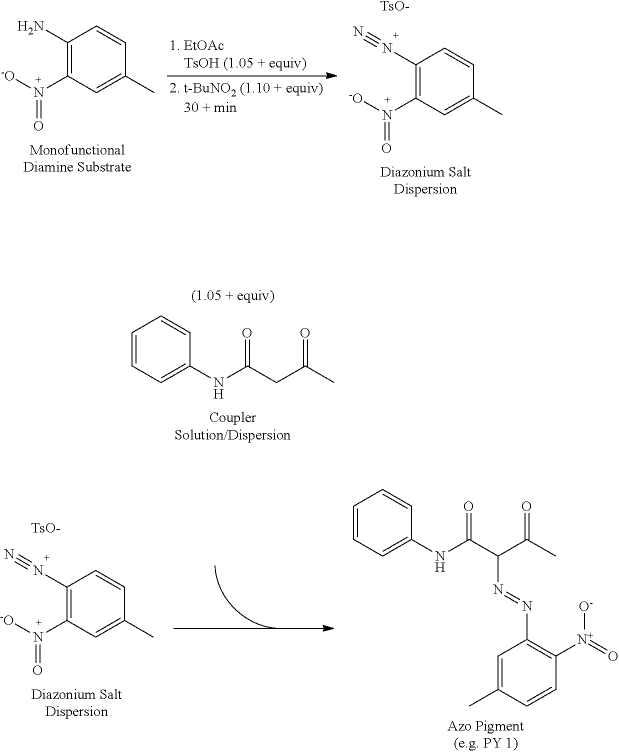

### Method B

A diazonium salt was generated when >0.4 mmol of a primary amine was reacted with >0.4 mmol of a nitrite ester in the presence of >0.4 mmol of an acid. Preferred results were obtained when the primary amine was used at 0.42 mmol with 0.46 mmol of the nitrite ester and 0.44 mmol of the acid in good mixing conditions. The diazonium salt was then reacted with 0.2 mmol of a coupler. An example of Method B is shown below:

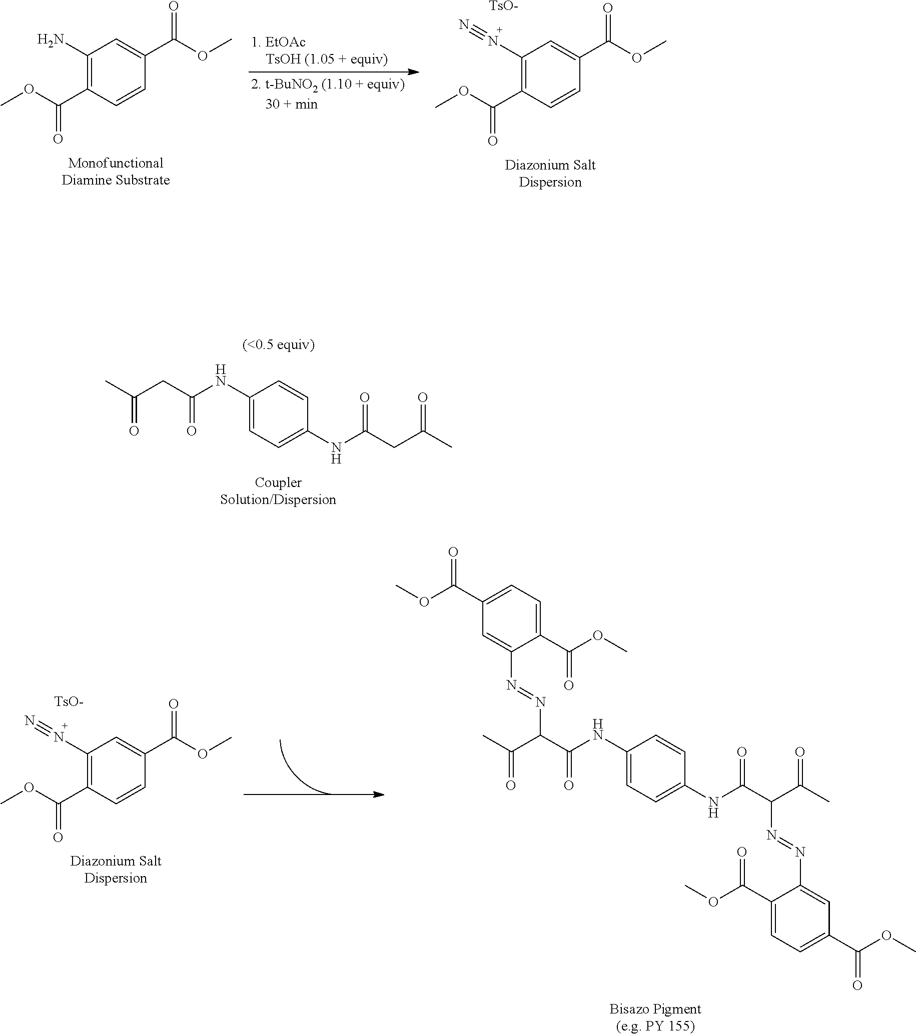

### Method C

A diazonium salt was generated when 0.2 mmol of a primary amine was reacted with >0.4 mmol of a nitrite ester in the presence of >0.4 mmol of an acid. Preferred results were obtained when the primary amine was used at 0.42 mmol with 0.46 mmol of the nitrite ester and 0.44 mmol of the acid in good mixing conditions. The diazonium salt was then reacted with >0.4 mmol of a coupler, with preferred results obtained with 0.42 mmol in good mixing conditions. An example of Method C is shown below:

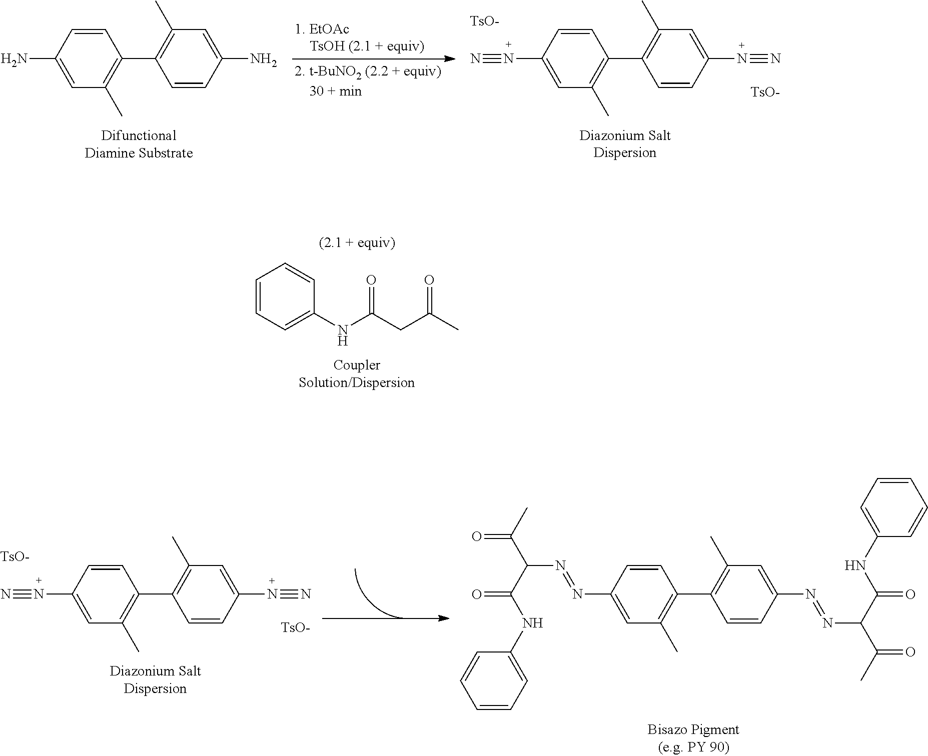

### EXAMPLE 2

This example demonstrates that the methods disclosed herein may be used to form a wide variety of pigments, including known pigments and novel pigments, in accordance with some embodiments.

Two different primary amines (3,3′-Dimethyl (1,1′-binaphthalene)-4,4′-diamine [13138-48-2] and 4,4″-diamino-p-terphenyl [3365-85-3]) were reacted with nitrite esters in the presence of an acid and an organic solvent to form diazonium salts, which were reacted with a variety of couplers to form pigments. The structures of the primary amines, couplers, and pigments are shown below, where Scheme 1 shows the couplers reacted with, and the pigments formed from, 3,3′-Dimethyl (1,1′-binaphthalene)-4,4′-diamine, and Scheme 2 shows the couplers reacted with, and the pigments formed from, 4,4″-diamino-p-terphenyl.

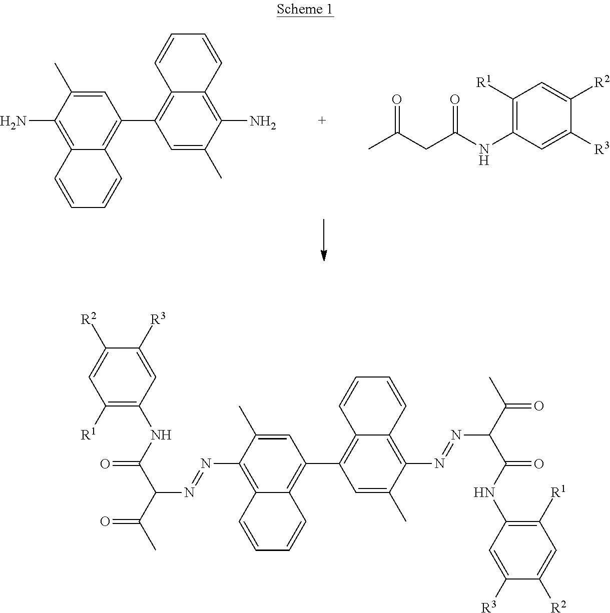

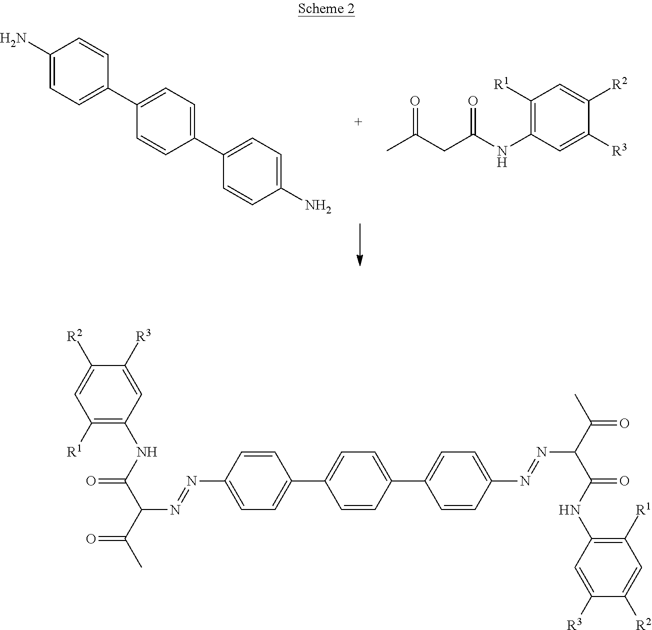

In Scheme 1 and Scheme 2, the couplers were as shown in Table 1.

TABLE 1
Couplers used in Scheme 1 and Scheme 2

| Coupling Compound | R¹ | R² | R³ |
|---|---|---|---|
| Acetoacetanalide | H | H | H |
| [102-01-2] | | | |
| N-(2-Methylphenyl)-3-oxobutanamide | CH₃ | H | H |
| [93-68-5] | | | |
| N-(4-Methylphenyl)-3-oxobutanamide | H | CH₃ | H |
| [2415-85-2] | | | |
| N-(2,4-Dimethylphenyl)-3-oxobutanamide | CH₃ | CH₃ | H |
| [97-36-9] | | | |
| N-(2-Methoxyphenyl)-3-oxobutanamide | OCH₃ | H | H |
| [92-15-9] | | | |
| N-(4-Methoxyphenyl)-3-oxobutanamide | H | OCH₃ | H |
| [5437-98-9] | | | |
| N-(4-Ethoxyphenyl)-3-oxobutanamide | H | OCH₂CH₃ | H |
| [122-82-7] | | | |
| N-(2,5-Dimethoxyphenyl)-3- | OCH₃ | H | OCH₃ |
| oxobutanamide | | | |
| [6375-27-5] | | | |

Examples of pigments obtained from Scheme 1 include the following:

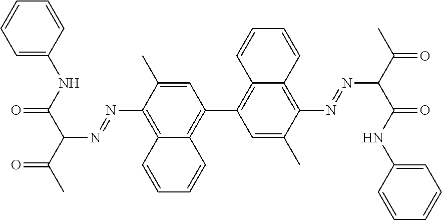 [SMILES: CC(=O)C(/N=N/c1c(C)cc(-c2cc(C)c(/N=N/C(C(C)=O)C(=O)Nc3ccccc3)c3ccccc23)c2ccccc12)C(=O)Nc1ccccc1]

N-Phenyl2-[(E)-{3,3′-dimethyl-4′-[(E)-1-(N-phenylcarbamoyl)-2-oxopropylazo]-1,1′-binaphthyl-4-yl}azo]acetoacetamide
O═C(NC(═CC═C1)C═C1)C(/N═N/C(C(C)═CC1C(C═C(C)C2/N═N/C(C(═O)NC(C═CC3)=CC=3)C(═O)C)═C(C═CC3)C=2C=3)=C(C═CC2)C=1C=2)C(═O)C;

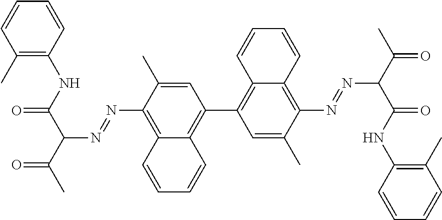 [SMILES: CC(=O)C(/N=N/c1c(C)cc(-c2cc(C)c(/N=N/C(C(C)=O)C(=O)Nc3ccccc3C)c3ccccc23)c2ccccc12)C(=O)Nc1ccccc1C]

N-o-Tolyl2-[(E)-{3,3′-dimethyl-4′-[(E)-1-(N-o-tolylcarbamoyl)-2-oxopropylazo]-1,1′-binaphthyl-4-yl}azo]acetoacetamide
O═C(NC(C═C1)=C(C)C═C1)C(/N═N/C(C(C)═CC1C(C═C(C)C2/N═N/C(C(C)═O)C(═O)NC(C═CC3)=C(C)C=3)=C(C=2C═C2)C═C2)=C(C=1C═C1)C═C1)C(═O)C;

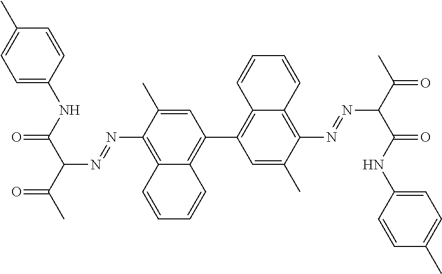 [SMILES: CC(=O)C(/N=N/c1c(C)cc(-c2cc(C)c(/N=N/C(C(C)=O)C(=O)Nc3ccc(C)cc3)c3ccccc23)c2ccccc12)C(=O)Nc1ccc(C)cc1]

N-p-Tolyl2-[(E)-{3,3′-dimethyl-4′-[(E)-1-(N-p-tolylcarbamoyl)-2-oxopropylazo]-1,1′-binaphthyl-4-yl}azo]acetoacetamide
O═C(NC(C═CC1C)═CC=1)C(/N═N/C(C(C)═CC1C(C═C2C)═C(C═CC3)C(C=3)=C2/N═N/C(C(═O)NC(C═CC2C)═CC=2)C(═O)C)═C(C=1C═C1)C═C1)C(═O)C;

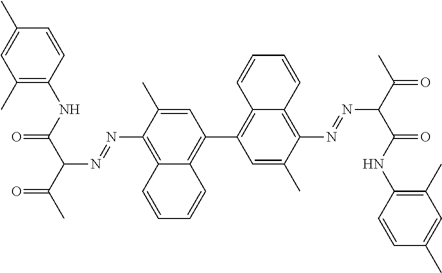 [SMILES: CC(=O)C(/N=N/c1c(C)cc(-c2cc(C)c(/N=N/C(C(C)=O)C(=O)Nc3ccc(C)cc3C)c3ccccc23)c2ccccc12)C(=O)Nc1ccc(C)cc1C]

N-2,4-Xylyl2-[(E)-{3,3′-dimethyl-4′-[(E)-1-(N-2,4-xylylcarbamoyl)-2-oxopropylazo]-1,1′-binaphthyl-4-yl}azo]acetoacetamide
O═C(C)C(/N═N/C(═C(C═C1)C(C═C1)=C1C(C═C2C)═C(C═CC3)C(C=3)=C2/N═N/C(C(C)═O)C(═O)NC(C═CC2C)═C(C=2)C)C(C)═C1)C(═O)NC(C═C1)=C(C)C═C1C;

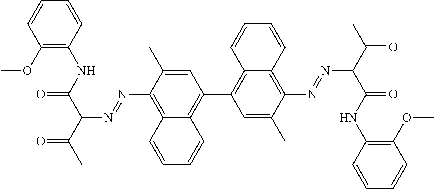

N-o-Methoxyphenyl2-[(E)-{3,3′-dimethyl-4′-[(E)-1-(N-o-methoxyphenylcarbamoyl)-2-oxopropylazo]-1,1′-binaphthyl-4-yl}azo]acetoacetamide
O═C(C)C(/N═N/C(C(C)═CC1C(C═C(C)C2/N═N/C(C(═O)C)C(═O)NC(C═CC3)=C(C=3)OC)═C(C=2C═C2)C═C2)=C(C=1C═C1)C═C1)C(═O)NC(C═C1)=C(OC)C═C1;

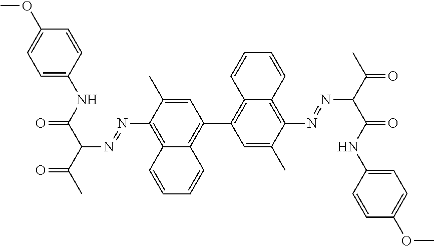

N-p-Methoxyphenyl2-[(E)-{3,3′-dimethyl-4′-[(E)-1-(N-p-methoxyphenylcarbamoyl)-2-oxopropylazo]-1,1′-binaphthyl-4-yl}azo]acetoacetamide
O═C(C)C(/N═N/C(═C(C═C1)C(C═C1)=C1C(C═C(C)C2/N═N/C(C(═O)C)C(═O)NC(C═CC3O C)═CC=3)=C(C=2C═C2)C═C2)C(C)═C1)C(═O)NC(C═CC1OC)═CC=1;

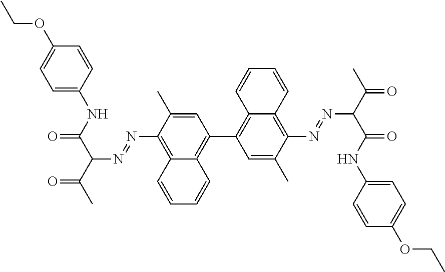 [SMILES: CCOc1ccc(NC(=O)C(/N=N/c2c(C)cc(-c3cc(C)c(/N=N/C(C(C)=O)C(=O)Nc4ccc(OCC)cc4)c4ccccc34)c3ccccc23)C(C)=O)cc1]

N-p-Ethoxyphenyl2-[(E)-{3,3′-dimethyl-4′-[(E)-1-(N-p-ethoxyphenylcarbamoyl)-2-oxopropylazo]-1,1′-binaphthyl-4-yl}azo]acetoacetamide
O═C(C)C(/N═N/C(═C(C═CC1)C(C=1)=C1C(C═C2C)═C(C═C3)C(C═C3)=C2/N═N/C(C(═O)NC(═CC═C2OCC)C═C2)C(C)═O)C(═C1)C)C(═O)NC(C═CC1OCC)═CC=1; and

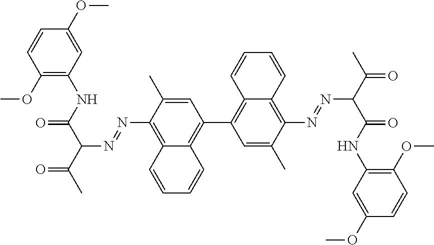

N-2,5-Dimethoxyphenyl2-[(E)-{3,3′-dimethyl-4′-[(E)-1-(N-2,5-dimethoxyphenylcarbamoyl)-2-oxopropylazo]-1,1′-binaphthyl-4-yl}azo]acetoacetamide
O═C(C)C(/N═N/C(C(C)═CC1C(C═C(C)C2/N═N/C(C(═O)NC(C═C3OC)═C(C═C3)OC) C(C)═O)═C(C=2C═C2)C═C2)=C(C═CC2)C=1C=2)C(═O)NC(C═C(C1)OC)═C(C=1)O C.

Examples of pigments obtained from Scheme 2 include the following:

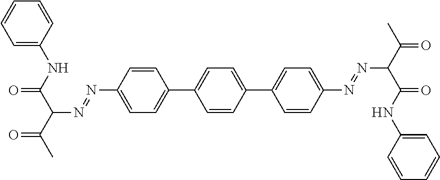 [SMILES: CC(=O)C(/N=N/c1ccc(-c2ccc(-c3ccc(/N=N/C(C(C)=O)C(=O)Nc4ccccc4)cc3)cc2)cc1)C(=O)Nc1ccccc1]

N-Phenyl3-oxo-2-[(E)-{4″-[(E)-1-(N-phenylcarbamoyl)-2-oxopropylazo]-p-terphenyl-4-yl}azo]butyramide
O═C(NC(═CC═C1)C═C1)C(/N═N/C(═CC═CIC(═CC═C2C(C═CC3/N═N/C(C(═O)NC(═CC═C4)C═C4)C(C)═O)═CC=3)C═C2)C═C1)C(═O)C;

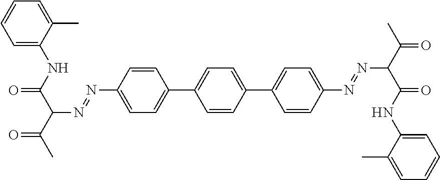 [SMILES: CC(=O)C(/N=N/c1ccc(-c2ccc(-c3ccc(/N=N/C(C(C)=O)C(=O)Nc4ccccc4C)cc3)cc2)cc1)C(=O)Nc1ccccc1C]

N-o-Tolyl3-oxo-2-[(E)-{4″-[(E)-1-(N-o-tolylcarbamoyl)-2-oxopropylazo]-p-terphenyl-4-yl}azo]butyramide
O═C(C)C(/N═N/C(═CC═C1C(═CC═C2C(═CC═C3/N═N/C(C(═O)C)C(═O)NC(═CC4)C(C)═CC=4)C═C3)C═C2)C═C1)C(═O)NC(═CC1)C(C)═CC=1;

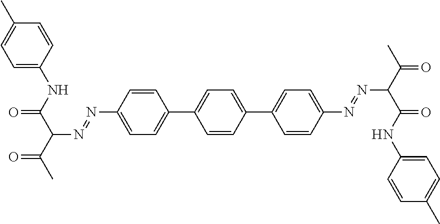 [SMILES: CC(=O)C(/N=N/c1ccc(-c2ccc(-c3ccc(/N=N/C(C(C)=O)C(=O)Nc4ccc(C)cc4)cc3)cc2)cc1)C(=O)Nc1ccc(C)cc1]

N-p-Tolyl3-oxo-2-[(E)-{4″-[(E)-1-(N-p-tolylcarbamoyl)-2-oxopropylazo]-p-terphenyl-4-yl}azo]butyramide
O═C(C)C(/N═N/C(═CC═C1C(C=CC2C(═CC═C3/N═N/C(C(═O)C)C(═O)NC(═CC═C4C)C═C4)C═C3)=CC=2)C═C1)C(═O)NC(═CC═C1C)C═C1;

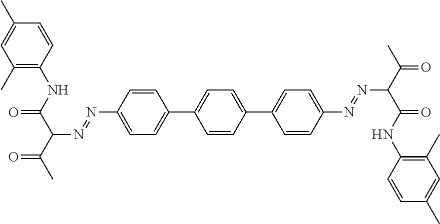 [SMILES: CC(=O)C(/N=N/c1ccc(-c2ccc(-c3ccc(/N=N/C(C(C)=O)C(=O)Nc4ccc(C)cc4C)cc3)cc2)cc1)C(=O)Nc1ccc(C)cc1C]

N-2,4-Xylyl3-oxo-2-[(E)-{4″-[(E)-1-(N-2,4-xylylcarbamoyl)-2-oxopropylazo]-p-terphenyl-4-yl}azo]butyramide
O═C(C)C(/N═N/C(═CC═C1C(═CC═C2C(═CC═C3/N═N/C(C(═O)C)C(═O)NC(C═CC4C)═C(C=4)C)C═C3)C═C2)C═C1)C(═O)NC(C═CC1C)═C(C=1) C;

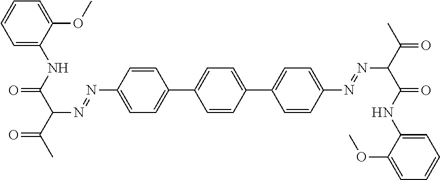 [SMILES: COc1ccccc1NC(=O)C(/N=N/c1ccc(-c2ccc(-c3ccc(/N=N/C(C(C)=O)C(=O)Nc4ccccc4OC)cc3)cc2)cc1)C(C)=O]

N-o-Methoxyphenyl3-oxo-2-[(E)-{4″-[(E)-1-(N-o-methoxyphenylcarbamoyl)-2-oxopropylazo]-p-terphenyl-4-yl}azo]butyramide
O═C(C)C(/N═N/C(═CC═C1C(═CC═C2C(C═CC3/N═N/C(C(═O)NC(═CC4)C(═CC=4) OC)C(═O)C)═CC=3)C═C2)C═C1)C(═O)NC(═CC1)C(OC)═CC=1;

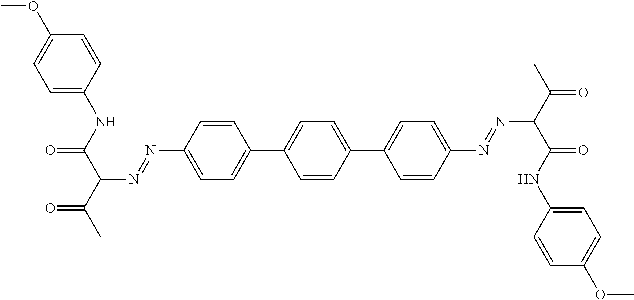

N-p-Methoxyphenyl3-oxo-2-[(E)-{4″-[(E)-1-(N-p-methoxyphenylcarbamoyl)-2-oxopropylazo]-p-terphenyl-4-yl}azo]butyramide
O═C(NC(C=CC1OC)═CC=1) C(/N═N/C(C═CC1C(C═CC2C(═CC═C3/N═N/C(C(═O)N C(C═CC4OC)═CC=4)C(C)═O)C═C3)=CC=2)=CC=1) C(C)═O;

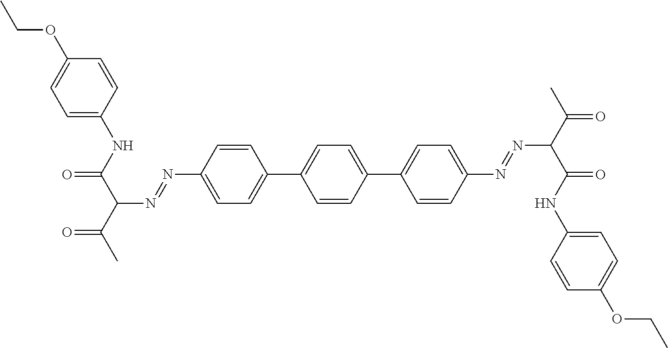 [SMILES: CCOc1ccc(NC(=O)C(/N=N/c2ccc(-c3ccc(-c4ccc(/N=N/C(C(C)=O)C(=O)Nc5ccc(OCC)cc5)cc4)cc3)cc2)C(C)=O)cc1]

N-p-Ethoxyphenyl3-oxo-2-[(E)-{4″-[(E)-1-(N-p-ethoxyphenylcarbamoyl)-2-oxopropylazo]-p-terphenyl-4-yl}azo]butyramide
O═C(C)C(/N═N/C(═CC═C1C(═CC═C2C(C═CC3/N═N/C(C(C)═O)C(═O)NC(═CC═C4 OCC)C═C4)=CC=3)C═C2)C═C1)C(═O)NC(═CC═C1OCC)C═C1; and

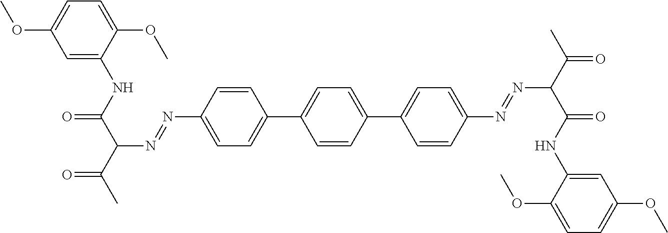

N-2,5-Dimethoxyphenyl3-oxo-2-[(E)-{4″-[(E)-1-(N-2,5-dimethoxyphenylcarbamoyl)-2-oxopropylazo]-p-terphenyl-4-yl}azo]butyramide
O═C(C)C(/N═N/C(C═CC1C(C═CC2C(═CC═C3/N═N/C(C(═O)C)C(═O)NC(═CC4OC) C(═CC=4)OC)C═C3)=CC=2)=CC=1) C(═O)NC(═CC1OC)C(OC)═CC=1.

3,3′-Dimethyl (1,1′-binaphthalene)-4,4′-diamine [13138-48-2] was again reacted with nitrite esters in the presence of an acid and an organic solvent to form diazonium salts, which were reacted with a variety of couplers to form pigments. The structures of the couplers and pigments are as shown in the Schemes below.

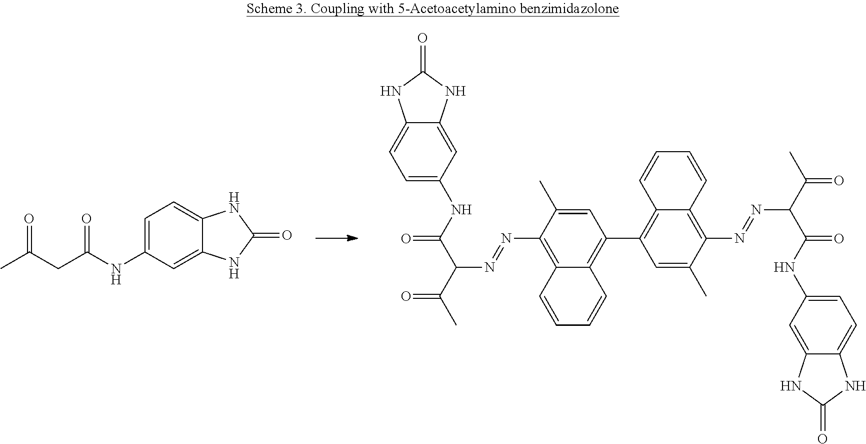 [SMILES: CC(=O)CC(=O)Nc1ccc2[nH]c(=O)[nH]c2c1 ; CC(=O)C(/N=N/c1c(C)cc(-c2cc(C)c(/N=N/C(C(C)=O)C(=O)Nc3ccc4[nH]c(=O)[nH]c4c3)c3ccccc23)c2ccccc12)C(=O)Nc1ccc2[nH]c(=O)[nH]c2c1]

N-(2-Oxo-2,3-dihydro-1H-1,3-benzimidazol-5-yl) 2-[(E)-{3,3′-dimethyl-4′-[(E)-1-(N-2-oxo-2,3-dihydro-1H-1,3-benzimidazol-5-ylcarbamoyl)-2-oxopropylazo]-1,1′-binaphthyl-4-yl}azo]acetoacetamide
O═C(NC(═CC═C1N2)C═C1NC2=O)C(/N═N/C(C(C)═CC1C(C═C(C)C2/N═N/C(C(C)═O)C(═O)NC(═CC═C3N4)C═C3NC4=O)═C(C═CC3)C=2C=3)=C(C=1C═C1)C═C1)C(═O)C

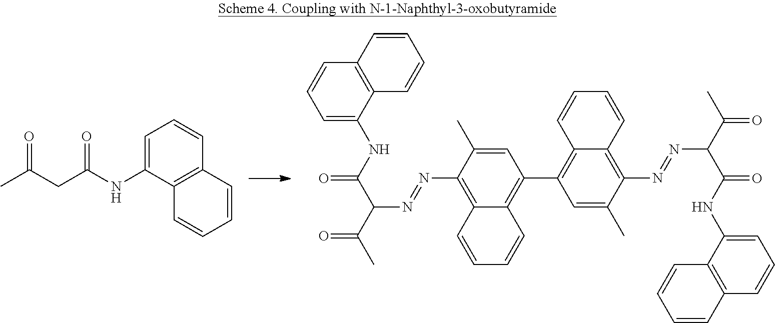 [SMILES: CC(=O)CC(=O)Nc1cccc2ccccc12 ; CC(=O)C(/N=N/c1c(C)cc(-c2cc(C)c(/N=N/C(C(C)=O)C(=O)Nc3cccc4ccccc34)c3ccccc23)c2ccccc12)C(=O)Nc1cccc2ccccc12]

N-1-Naphthyl2-[(E)-{3,3′-dimethyl-4′-[(E)-1-(N-1-naphthylcarbamoyl)-2-oxopropylazo]-1,1′-binaphthyl-4-yl}azo]acetoacetamide
O═C(C)C(/N═N/C(C(C)═CC1C(C═C2C)═C(C═C3)C(═C2/N═N/C(C(C)═O)C(═O)NC(═CC═C2)C(═C2C═C2)C═C2)C═C3)=C(C=1C═C1)C═C1)C(═O)NC(═CC═C1)C(═C1C=C1)C═C1

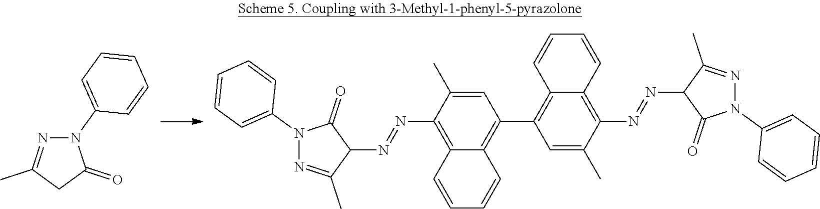 [SMILES: CC1=NN(c2ccccc2)C(=O)C1 ; CC1=NN(c2ccccc2)C(=O)C1/N=N/c1c(C)cc(-c2cc(C)c(/N=N/C3C(=O)N(c4ccccc4)N=C3C)c3ccccc23)c2ccccc12]

4-[(E)-{3,3′-Dimethyl-4′-[(E)-3-methyl-5-oxo-1-phenyl-2-pyrazolin-4-ylazo]-1,1′-binaphthyl-4-yl}azo]-3-methyl-1-phenyl-2-pyrazolin-5-one
O═C(N(N1) C(C═CC2)=CC=2)C(/N═N/C(═C(C═CC2)C(C=2)=C2C(C═C3C)═C(C═C4) C(═C3/N═N/C(C(═N3)C)C(═O)N3C(═CC═C3)C═C3)C═C4)C(C)═C2)C=1C

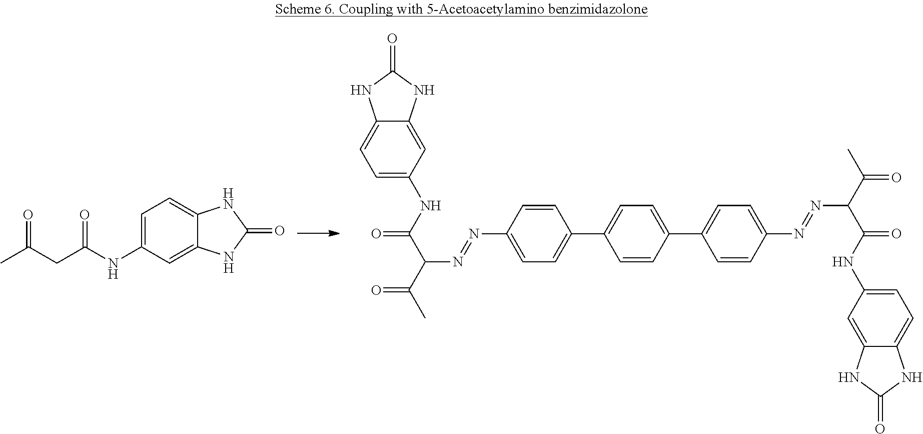 [SMILES: CC(=O)CC(=O)Nc1ccc2[nH]c(=O)[nH]c2c1 ; CC(=O)C(/N=N/c1ccc(-c2ccc(-c3ccc(/N=N/C(C(C)=O)C(=O)Nc4ccc5[nH]c(=O)[nH]c5c4)cc3)cc2)cc1)C(=O)Nc1ccc2[nH]c(=O)[nH]c2c1]

N-(2-Oxo-2,3-dihydro-1H-1,3-benzimidazol-5-yl) 3-oxo-2-[(E)-{4″-[(E)-1-(N-2-oxo-2,3-dihydro-1H-1,3-benzimidazol-5-ylcarbamoyl)-2-oxopropylazo]-p-terphenyl-4-yl}azo]butyramide
O═C(NC(═CC═C1N2)C═C1NC2=O)C(/N═N/C(═CC═C1C(C═CC2C(C═CC3/N═N/C(C(═O)NC(C═C(C4N5)NC5=O)═CC=4)C(═O)C)═CC=3)=CC=2)C═C1)C(═O)C

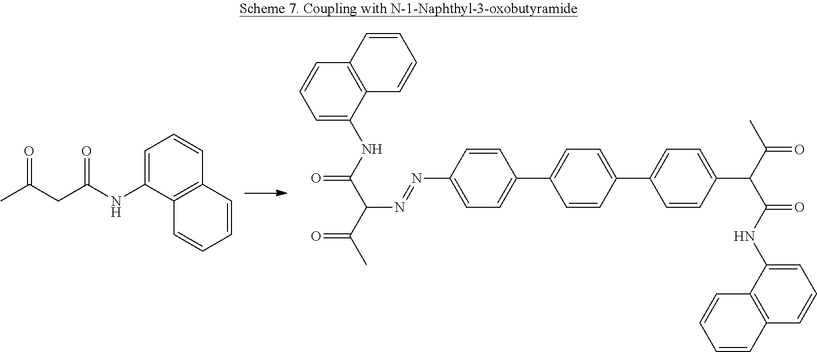 [SMILES: CC(=O)CC(=O)Nc1cccc2ccccc12 ; CC(=O)C(/N=N/c1ccc(-c2ccc(-c3ccc(C(C(C)=O)C(=O)Nc4cccc5ccccc45)cc3)cc2)cc1)C(=O)Nc1cccc2ccccc12]

N-1-Naphthyl3-oxo-2-[(E)-{4″-[1-(N-1-naphthylcarbamoyl)-2-oxopropyl]-p-terphenyl-4-yl}azo]butyramide
O═C(C)C(/N═N/C(═CC═C1C(C═CC2C(C═CC3C(C(═O)NC(═CC4)C(C═CC5)=C(C=5) C=4)C(C)=O)═CC=3)=CC=2)C═C1)C(═O)NC(═CC1)C(C═CC2)=C(C=2)C=1

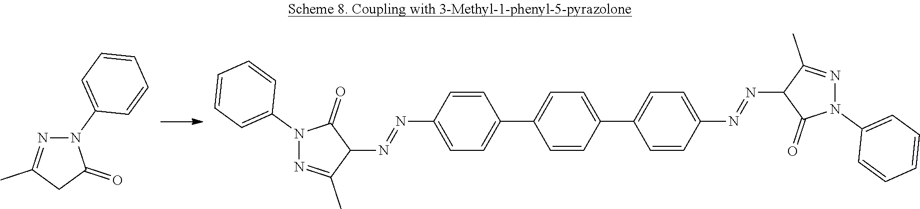 [SMILES: CC1=NN(c2ccccc2)C(=O)C1 ; CC1=NN(c2ccccc2)C(=O)C1/N=N/c1ccc(-c2ccc(-c3ccc(/N=N/C4C(=O)N(c5ccccc5)N=C4C)cc3)cc2)cc1]

3-Methyl-4-[(E)-{4″-[(E)-3-methyl-5-oxo-1-phenyl-2-pyrazolin-4-ylazo]-p-terphenyl-4-yl}azo]-1-phenyl-2-pyrazolin-5-one
O═C(C(C1C)/N═N/C(C═CC2C(C═CC3C(═CC═C4/N═N/C(C(C)═N5) C(═O)N5C(C═C C5)=CC=5) C═C4)=CC=3)=CC=2) N(N=1) C(C═CC1)=CC=1

### EXAMPLE 3

This example demonstrates various methods used to prepare various types of couplers.

### Coupler Method A

The coupler comprised only one labile hydrogen bonded to carbon and/or only equivalent labile hydrogens (e.g., two equivalent labile hydrogens) bonded to carbon (e.g., bonded to the same carbon). The coupler was dissolved or dispersed in 2M sodium hydroxide at a concentration of 0.3 M or less.

### Coupler Method B

The coupler comprised two or more inequivalent labile hydrogens bonded to carbon. The coupler was dissolved or dispersed in 2M sodium hydroxide at a concentration of 0.3 M or less. The pH of the dispersion was adjusted to 5 using a 10% acetic acid solution under constant stirring.

### Coupler Method C

A stock solution of calcium bicarbonate was prepared by dispersing 20 g of calcium carbonate in 2 L of carbonated water. After mixing overnight, the pH was confirmed to be approximately 7 and some settling behavior was seen. This material was dispersed back into the calcium bicarbonate solution before each use, and the coupler was dissolved or dispersed at a concentration of 0.3 M or less. This coupler procedure was used in a method of producing a calcium pigment lake.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of forming a pigment, comprising:
reacting a primary amine and a nitrite ester in the presence of an acid and an organic solvent to form a diazonium salt; and
forming a pigment by reacting the diazonium salt with a coupler in a biphasic mixture comprising the organic solvent and water.

2. The method of claim 1, wherein the method does not involve any halogenated compounds.

3-7. (canceled)

8. The method of claim 1, wherein the organic solvent is immiscible with water.

9. The method of claim 1, wherein the organic solvent comprises an ester.

10. The method of claim 9, wherein the ester comprises ethyl acetate, n-butyl acetate, t-butyl acetate, n-hexyl acetate, and/or n-octyl acetate.

11. (canceled)

12. The method of claim 1, wherein the acid comprises a monoprotic strong organic acid.

13. The method of claim 1, wherein the acid comprises an aromatic sulfonic acid.

14. The method of claim 1, wherein the acid comprises benzenesulfonic acid and/or p-toluenesulfonic acid.

15-17. (canceled)

18. The method of claim 1, wherein the nitrite ester comprises methyl nitrite, ethyl nitrite, n-propyl nitrite, isopropyl nitrite, n-butyl nitrite, sec-butyl nitrite, and/or tert-butyl nitrite.

19-21. (canceled)

22. The method of claim 1, wherein the coupler comprises one or more acidic carbons that can serve as a nucleophile, wherein an acidic carbon is a C—H bond that has a labile hydrogen and a pKa value of less than or equal to 30.

23-29. (canceled)

30. The method of claim 1, wherein the method further comprises adding a surfactant to the diazonium salt.

31. The method of claim 30, wherein the surfactant comprises a water-in-oil emulsifier and/or has a hydrophilic-lipophilic balance (HLB) between 4-6.

32. The method of claim 30, wherein the surfactant comprises a fatty acid ester.

33. The method of claim 30, wherein the surfactant comprises sorbitan monooleate and/or n-octyl pyrrolidone.

34. (canceled)

35. The method of claim 1, wherein the method further comprises adding an amphiphilic solvent to the diazonium salt.

36. The method of claim 35, wherein the amphiphilic solvent is water immiscible and/or has a chain length of greater than or equal to 6 and less than or equal to 12.

37. The method of claim 35, wherein the amphiphilic solvent comprises n-octyl-pyrrolidone, n-hexyl acetate, and/or n-octyl acetate.

38. (canceled)

39. The method of claim 1, wherein the method comprises reacting the diazonium salt with the coupler without first isolating the diazonium salt.

40. (canceled)

41. The method of claim 1, wherein the method further comprises isolating the pigment; and
wherein isolating the pigment comprises washing, filtering, extracting, and/or centrifuging the pigment.

42-45. (canceled)

46. The method of claim 1, wherein the pigment comprises an azo pigment, a monoazo pigment, a bisazo pigment, and/or a pigment lake.

47-51. (canceled)

52. A pigment, comprising: 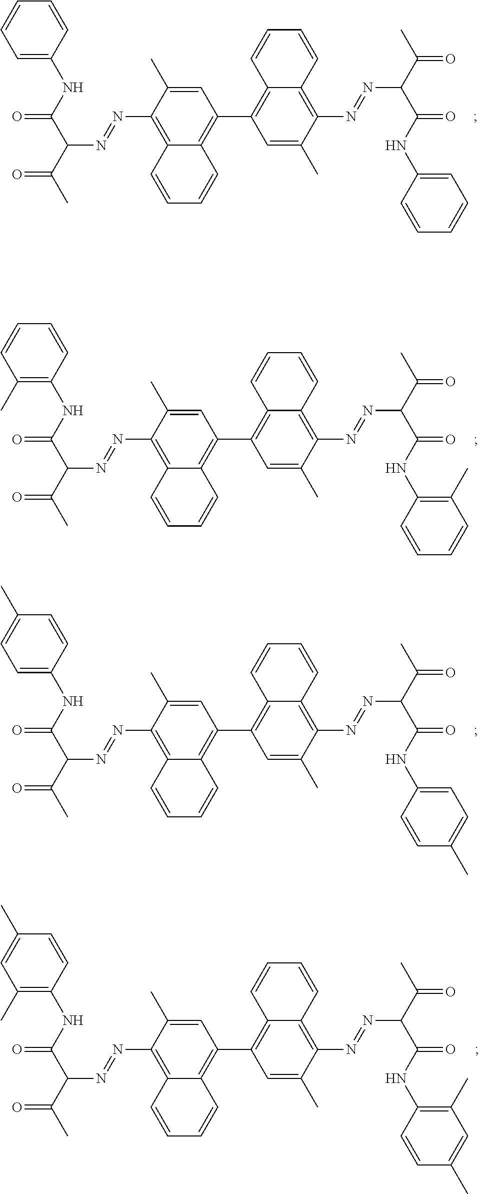 [SMILES: CC(=O)C(/N=N/c1c(C)cc(-c2cc(C)c(/N=N/C(C(C)=O)C(=O)Nc3ccccc3)c3ccccc23)c2ccccc12)C(=O)Nc1ccccc1 ; CC(=O)C(/N=N/c1c(C)cc(-c2cc(C)c(/N=N/C(C(C)=O)C(=O)Nc3ccccc3C)c3ccccc23)c2ccccc12)C(=O)Nc1ccccc1C ; CC(=O)C(/N=N/c1c(C)cc(-c2cc(C)c(/N=N/C(C(C)=O)C(=O)Nc3ccc(C)cc3)c3ccccc23)c2ccccc12)C(=O)Nc1ccc(C)cc1 ; CC(=O)C(/N=N/c1c(C)cc(-c2cc(C)c(/N=N/C(C(C)=O)C(=O)Nc3ccc(C)cc3C)c3ccccc23)c2ccccc12)C(=O)Nc1ccc(C)cc1C] 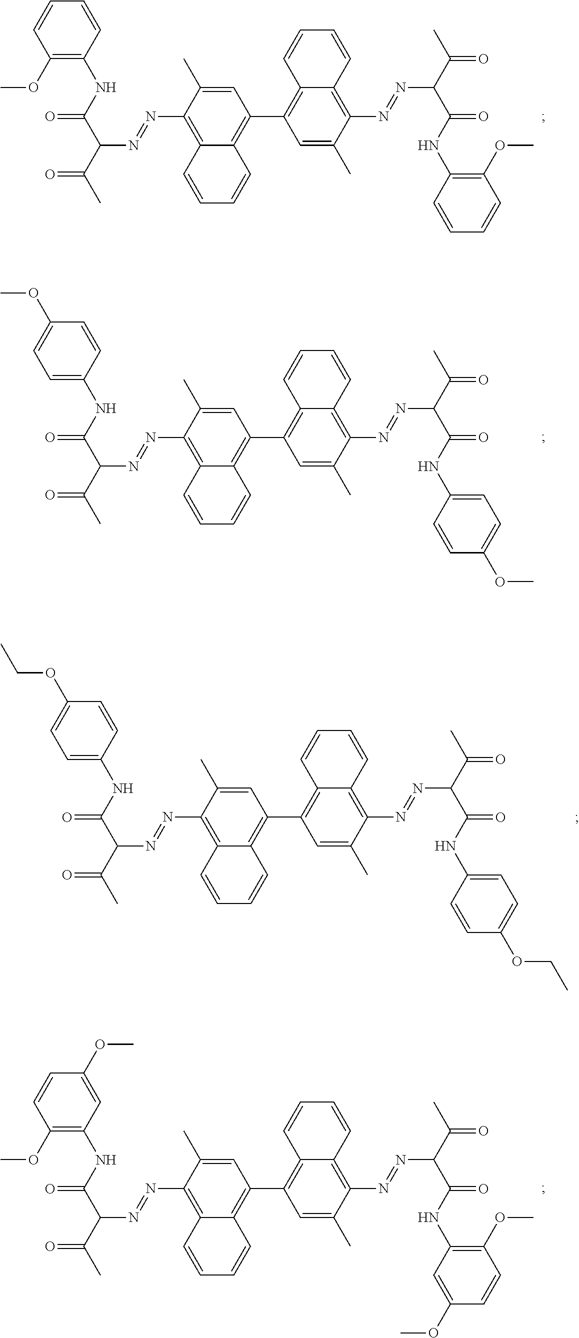 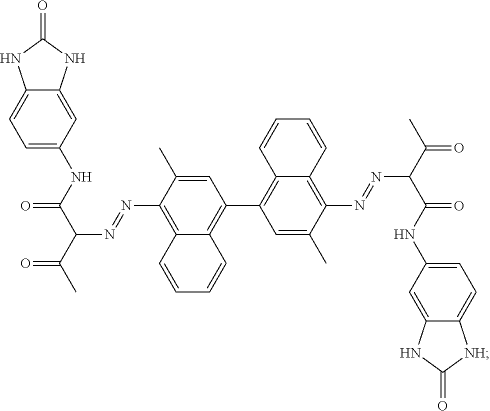 [SMILES: CC(=O)C(/N=N/c1c(C)cc(-c2cc(C)c(/N=N/C(C(C)=O)C(=O)Nc3ccc4[nH]c(=O)[nH]c4c3)c3ccccc23)c2ccccc12)C(=O)Nc1ccc2[nH]c(=O)[nH]c2c1] 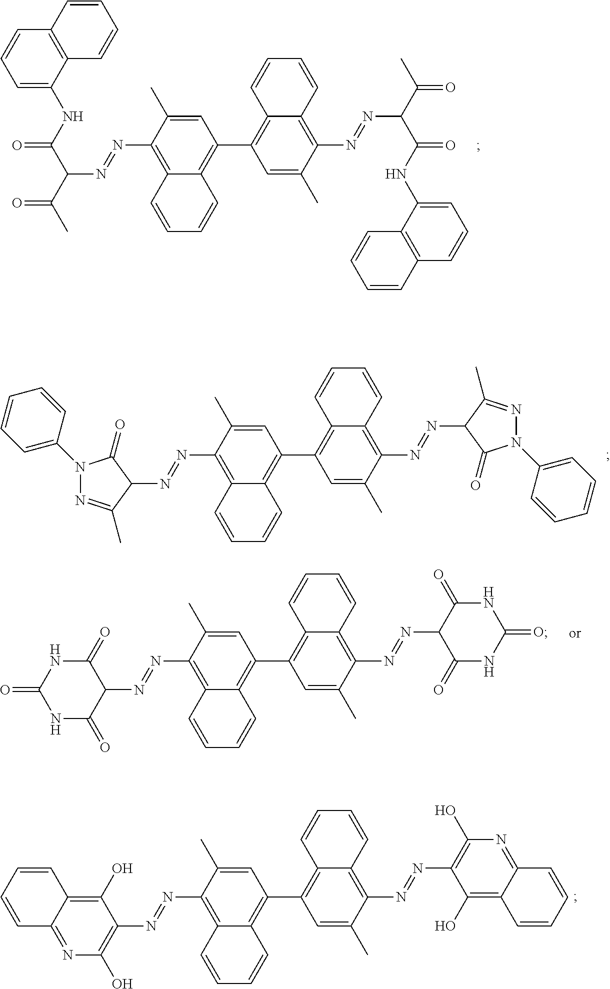 [SMILES: CC(=O)C(/N=N/c1c(C)cc(-c2cc(C)c(/N=N/C(C(C)=O)C(=O)Nc3cccc4ccccc34)c3ccccc23)c2ccccc12)C(=O)Nc1cccc2ccccc12 ; CC1=NN(c2ccccc2)C(=O)C1/N=N/c1c(C)cc(-c2cc(C)c(/N=N/C3C(=O)N(c4ccccc4)N=C3C)c3ccccc23)c2ccccc12 ; Cc1cc(-c2cc(C)c(/N=N/C3C(=O)NC(=O)NC3=O)c3ccccc23)c2ccccc2c1/N=N/C1C(=O)NC(=O)NC1=O ; Cc1cc(-c2cc(C)c(/N=N/c3c(O)nc4ccccc4c3O)c3ccccc23)c2ccccc2c1/N=N/c1c(O)nc2ccccc2c1O]
or a tautomer or polymorph thereof.

53. A pigment, comprising: 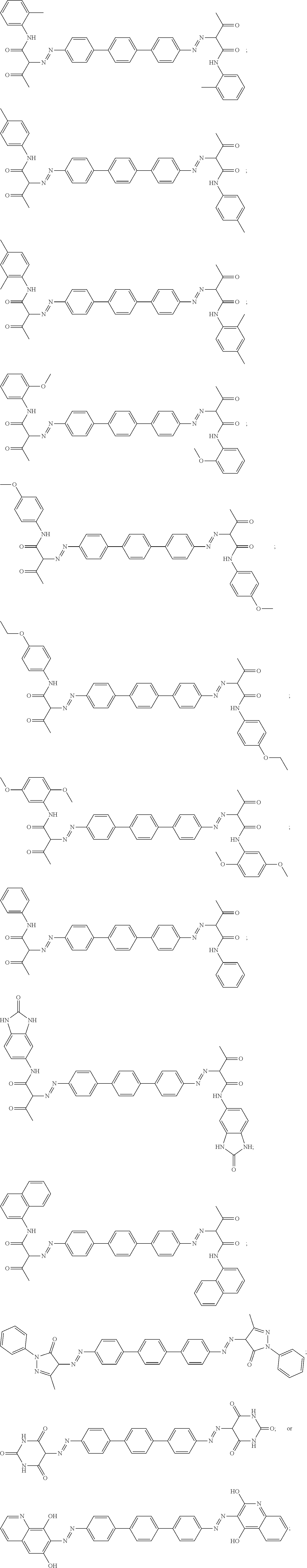
or a tautomer or polymorph thereof.

54. (canceled)
